(19) United States
(12) Patent Application Publication
(10) Pub. No.: US 2024/0299640 A1
(43) Pub. Date: Sep. 12, 2024

(54) Drug Primary Package Having Integrated RFID Tag

(71) Applicant: SiO2 Medical Products, Inc., Auburn, AL (US)

(72) Inventors: Robert S. Abrams, Albany, NY (US); Yves Steffen, Auburn, AL (US)

(21) Appl. No.: 18/560,187

(22) PCT Filed: May 11, 2022
(86) PCT No.: PCT/US2022/028853

§ 371 Date: Nov. 10, 2023

Related U.S. Application Data
(60) Provisional application No. 63/215,879, filed on Jun. 28, 2021.
(60) Provisional application No. 63/187,385, filed on May 11, 2021.

(51) Int. Cl. A61M 5/00

(52) U.S. Cl. CPC A61M 5/002; A61M 2205/3368; A61M 2205/3584; A61M 2205/60

(57) ABSTRACT

The present disclosure is directed to plastic pharmaceutical vessels, such as vials, pre-filled syringes, and cartridges having an electronic component, such as an RFID tag, embedded in and fully enclosed by a wall thereof. The electronic component may be positioned at a specific site that allows for the component to be concealed by a conventional closure, such as in the neck portion of a vial or the needle or Luer hub of a syringe barrel. The present disclosure is also directed to methods of preparing and using pharmaceutical vessels having an electronic component incorporated therein.

The present application claims priority to U.S. Provisional Patent Application No. 63/187,385, filed on May 11, 2021, and U.S. Provisional Patent Application No. 63/215,879, filed on Jun. 28, 2021, the entireties of which are incorporated herein by reference.

### FIELD OF THE INVENTION

The traceability of pharmaceutical packages has become increasingly important. In an ideal scenario, a pharmaceutical package such as a drug-containing vial, cartridge, or pre-filled syringe, would be traceable across a timeline that includes (1) the manufacture of the container, (2) the filling of the container with a pharmaceutical product, and (3) the supply chain of the filled pharmaceutical package. The traceability of individual packages is a key feature to enable better control of the pharmaceutical throughout the supply chain.

### BACKGROUND OF THE INVENTION

To date, it has been suggested that an RFID tag may be attached to the outside surface of a vial as a “flag tag” or other exterior element, such as one that is attached to the metal crimp of the vial closure, see U.S. Pat. No. 9,589,226 B2, as an insert into the bottom face of a specially designed vial, see U.S. Pat. App. Pub. No. 2011/0199187 A1, or as a part of the vial lid, see U.S. Pat. App. Pub. No. 2019/0026616 A1. Similarly, it has been suggested that an RFID tag may be incorporated into a rigid needle shield that is attached to a syringe, see U.S. Pat. App. Pub. No. 2019/0328485 A1. Notably, most of these options would only allow for the tracking of the pharmaceutical package after the container was filled and sealed. Many of these options also require specially designed components, which are likely to be costly to manufacture and unlikely find widespread adoption. Finally, RFID tags located on the exterior of a vessel may easily be damaged.

### SUMMARY OF THE INVENTION

The present disclosure relates to pharmaceutical packages, such as vials, pre-filled syringes, cartridges, and other drug delivery devices, that incorporate an electronic component to provide traceability throughout a full value chain that includes (1) the manufacture of the container, (2) the filling of the container with a pharmaceutical product, and (3) the supply chain of the filled pharmaceutical package. The electronic component may be attached directly to the vessel that is filled and sealed to create a primary pharmaceutical package, e.g. to a vial body, a syringe barrel, or the body/barrel of a cartridge. In some embodiments, the electronic component may be embedded in the vessel, e.g. molded into a wall of a thermoplastic vessel, so that the electronic component is integral to the final pharmaceutical package and optionally not exposed on the exterior of the package, the interior of the package, or both.

Embodiments of the present disclosure are directed to a pharmaceutical package comprising: a vessel defining a lumen, a pharmaceutical solution within the lumen, a closure, and an electronic component attached to the vessel and configured to provide the package with the ability to be traced; and/or detect and/or record compliance or non-compliance with one or more storage conditions; and/or provide information regarding one or more administration parameters.

The pharmaceutical package of any embodiment, wherein the vessel is a syringe barrel, a cartridge, or a vial.

The pharmaceutical package of any embodiment, wherein the electronic component is an RFID tag, optionally an RFID tag that utilizes one or more frequencies within the range of 865 to 928 MHz, optionally an RFID tag that utilizes a frequency of about 13.56 MHz.

The pharmaceutical package of any embodiment, wherein the electronic component comprises an integrated circuit.

The pharmaceutical package of any embodiment, wherein the electronic component comprises data storage.

The pharmaceutical package of any embodiment, wherein the vessel comprises at least one wall made of a thermoplastic material.

The pharmaceutical package of any embodiment, wherein the thermoplastic material comprises one or more of the following: polypropylene, polyethylene, COP, COC, or CBC.

The pharmaceutical package of any embodiment, wherein the electronic component is embedded in a thermoplastic wall of the vessel.

The pharmaceutical package of any embodiment, wherein the electronic component is overmolded into a wall of the vessel.

The pharmaceutical package of any embodiment, wherein the thermoplastic material that makes up at least a portion of the vessel completely or substantially completely surrounds the electronic component.

The pharmaceutical package of any embodiment, wherein no part of the electronic component is on the exterior surface of the vessel.

The pharmaceutical package of any embodiment, wherein the package is a pre-filled syringe and wherein the electronic component is embedded in a hub portion of the syringe barrel.

The pharmaceutical package of any embodiment, wherein the package is a pre-filled syringe and wherein the electronic component is embedded in a transition region between a main body of the syringe barrel and a hub portion of the syringe barrel.

The pharmaceutical package of any embodiment, wherein the hub portion is a needle hub or a Luer hub.

The pharmaceutical package of any embodiment, wherein the closure comprises a rigid needle shield or a Luer cap, and wherein the electronic component is visually concealed by the closure.

The pharmaceutical package of any embodiment, wherein the package is a pre-filled syringe and wherein the electronic component is embedded in a main body of the syringe barrel adjacent a rear flange.

The pharmaceutical package of any embodiment, wherein the package is a filled vial, and wherein the electronic component is embedded in a neck portion of the vial, optionally in a thickened flange region.

The pharmaceutical package of any embodiment, wherein the closure comprises a stopper and a crimp, and wherein the electronic component is visually concealed by the closure.

The pharmaceutical package of any embodiment, wherein the package is a filled cartridge, and wherein the electronic component is embedded in a needle attachment portion of the cartridge.

The pharmaceutical package of any embodiment, wherein the closure comprises a cap and the electronic component is visually concealed by the closure.

The pharmaceutical package of any embodiment, further comprising a label and wherein the electronic component is embedded in a portion of the vessel that is visually concealed by the label.

The pharmaceutical package of any embodiment, wherein the package is a filled cartridge and the electronic component is configured to be directly read by a delivery device, optionally an auto-injector or an injection pen.

The pharmaceutical package of any embodiment, wherein the electronic component is visually concealed by a portion of the package.

The pharmaceutical package of any embodiment, wherein the package is configured so that the vessel may also be traced prior to filling of the lumen with the pharmaceutical solution and sealing of the lumen with the closure.

The pharmaceutical package of any embodiment, wherein the electronic component is detectable by automated visual inspection of the vessel in an empty state.

The pharmaceutical package of any embodiment, wherein the electronic component is configured to be written to using an external writer.

The pharmaceutical package of any embodiment, wherein the pharmaceutical solution is an injectable drug-containing solution.

The pharmaceutical package of any embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

The pharmaceutical package of any embodiment, wherein the electronic component has a thickness of 500 microns or less, optionally 400 microns or less, optionally 300 microns or less, optionally 200 microns or less, optionally 100 microns or less, optionally 50 microns or less.

The pharmaceutical package of any embodiment, wherein the pharmaceutical package has been subjected to sterilization, optionally sterilization by irradiation, optionally sterilization by gas.

The pharmaceutical package of any embodiment, wherein the electronic component is configured to withstand sterilization of the pharmaceutical package, optionally sterilization by irradiation, optionally sterilization by gas.

The pharmaceutical package of any embodiment, in which the electronic component comprises a temperature sensor, optionally in which the electronic component is a passive RFID temperature sensor.

The pharmaceutical package of any embodiment, in which the pharmaceutical solution within the lumen must be maintained within a defined temperature range and the electronic component is configured to detect and register a deviation from that temperature range.

The pharmaceutical package of any embodiment, in which the electronic component comprises a pressure sensor, optionally in which the electronic component is a passive RFID pressure sensor.

The pharmaceutical package of any embodiment, in which the pharmaceutical solution within the lumen must be maintained within a defined pressure range and the electronic component is configured to detect and register a deviation from that pressure range.

The pharmaceutical package of any embodiment, in which the pharmaceutical solution is to be administered at a defined dispense rate or within a defined range of acceptable dispense rates and the electronic component contains information regarding the defined dispense rate or defined range of acceptable dispense rates.

The pharmaceutical package of any embodiment, in which the information regarding the defined dispense rate or defined range of acceptable dispense rates is configured to be read by a delivery device, optionally an auto-injector or injection pen.

The pharmaceutical package of any embodiment, in which the pharmaceutical solution is to be administered using a defined needle insertion depth or defined range of acceptable needle insertion depths and the electronic component contains information regarding the defined needle insertion depth or defined range of acceptable needle insertion depths.

The pharmaceutical package of any embodiment, in which the information regarding the defined needle insertion depth or defined range of acceptable needle insertion depths is configured to be read by a delivery device, optionally an auto-injector or injection pen.

The pharmaceutical package of any embodiment, in which the electronic component is configured to register an interrogation event into a database.

An auto-injector or injection pen comprising the cartridge or syringe of any embodiment, wherein the auto-injector or injection pen comprises a reader configured to read the electronic component of the cartridge or syringe.

The auto-injector or injection pen of any embodiment, wherein the auto-injector or injection pen is configured to adjust one or more injection settings in response to information obtained from the electronic component.

The pharmaceutical package of any embodiment, in which the inner surface, the outer surface, or both of the wall in which the electronic component is embedded are identical or substantially identical to those of the wall of the same vessel prepared without the electronic component.

The pharmaceutical package of any embodiment, in which the inner surface, the outer surface, or both of the wall in which the electronic component is embedded are free of surface irregularities, such as those caused by insertion of an electronic component.

The pharmaceutical package of any embodiment, in which the electronic component comprises information, optionally identification information, stored directly on the component.

The pharmaceutical package of any embodiment, in which the electronic component is readable without connection to a computing network such as a cloud-computing network.

The pharmaceutical package of any embodiment, in which the electronic component is writable without connection to a computing network such as a cloud-computing network.

Embodiments of the present disclosure are also directed to a vessel having an electronic component attached to the vessel and configured to provide both the unfilled vessel and the pharmaceutical package that results from filling a lumen of the vessel with a pharmaceutical solution and then sealing the lumen with the ability to be traced. The vessel may be a syringe barrel, a vial, or a cartridge.

Embodiments of the present disclosure are directed to a syringe barrel having an electronic component attached to the syringe barrel and configured to provide both the unfilled syringe barrel and the pharmaceutical package that results from filling a lumen of the syringe barrel with a pharmaceutical solution and then sealing the lumen with the ability to be traced.

Embodiments of the present disclosure are directed to a vial having an electronic component attached to the vial and configured to provide both the unfilled vial and the pharmaceutical package that results from filling a lumen of the vial with a pharmaceutical solution and then sealing the lumen with the ability to be traced.

Embodiments of the present disclosure are directed to an injection cartridge having an electronic component attached to the cartridge and configured to provide both the unfilled cartridge and the pharmaceutical package that results from filling a lumen of the cartridge with a pharmaceutical solution and then sealing the lumen with the ability to be traced.

The vessel, syringe barrel, vial or cartridge of any embodiment, wherein the electronic component is an RFID tag, optionally an RFID tag that utilizes one or more frequencies within the range of 865 to 928 MHz, optionally an RFID tag that utilizes a frequency of about 13.56 MHz.

The vessel, syringe barrel, vial or cartridge of any embodiment, wherein the electronic component comprises an integrated circuit.

The vessel, syringe barrel, vial or cartridge of any embodiment, wherein the electronic component comprises data storage.

The vessel, syringe barrel, vial or cartridge of any embodiment, wherein the vessel, syringe barrel, vial or cartridge comprises at least one wall made of a thermoplastic material.

The vessel, syringe barrel, vial or cartridge of any embodiment, wherein the thermoplastic material comprises one or more of the following: polypropylene, polyethylene, COP, COC, or CBC.

The vessel, syringe barrel, vial or cartridge of any embodiment, wherein the electronic component is embedded in a thermoplastic wall of the vessel, syringe barrel, vial or cartridge.

The vessel, syringe barrel, vial or cartridge of any embodiment, wherein the electronic component is overmolded into a wall of the vessel, syringe barrel, vial or cartridge.

The vessel, syringe barrel, vial or cartridge of any embodiment, wherein the thermoplastic material that makes up at least a portion of the vessel, syringe barrel, vial or cartridge completely or substantially completely surrounds the electronic component.

The vessel, syringe barrel, vial or cartridge of any embodiment, wherein no part of the electronic component is on the exterior surface of the vessel, syringe barrel, vial or cartridge.

The syringe barrel of any embodiment, wherein the electronic component is embedded in a hub portion of the syringe barrel.

The syringe barrel of any embodiment, wherein the electronic component is embedded in a transition region between a main body of the syringe barrel and a hub portion of the syringe barrel.

The syringe barrel of any embodiment, wherein the hub portion is a needle hub or a Luer hub.

The syringe barrel of any embodiment, wherein the syringe barrel is configured so that the electronic component is visually concealed by a rigid needle shield or Luer cap.

The syringe barrel of any embodiment, wherein the electronic component is embedded in a main body of the syringe barrel adjacent a rear flange.

The vial of any embodiment, wherein the electronic component is embedded in a neck portion of the vial, optionally in a thickened flange region.

The vial of any embodiment, wherein the vial is configured so that the electronic component is visually concealed by a stopper and crimp closure.

The cartridge of any embodiment, wherein the electronic component is embedded in a needle attachment portion of the cartridge.

The cartridge of any embodiment, wherein the cartridge is configured so that the electronic component is visually concealed by a cap placed on the needle attachment portion.

The vessel, syringe barrel, vial or cartridge of any embodiment, further comprising a label and wherein the electronic component is embedded in a portion of the vessel, syringe barrel, vial or cartridge that is visually concealed by the label.

The cartridge of any embodiment, wherein cartridge is configured so that the electronic component is directly readable by a delivery device, optionally an auto-injector or an injection pen.

The vessel, syringe barrel, vial or cartridge of any embodiment, wherein the vessel, syringe barrel, vial or cartridge is configured so that the electronic component is visually concealed by a portion of the finished pharmaceutical package.

The vessel, syringe barrel, vial or cartridge of any embodiment, wherein the electronic component is detectable by automated visual inspection of the vessel, syringe barrel, vial or cartridge in an empty state.

The vessel, syringe barrel, vial or cartridge of any embodiment, wherein the electronic component is configured to be written to using an external writer.

The vessel, syringe barrel, vial or cartridge of any embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

The vessel, syringe barrel, vial or cartridge of any embodiment, wherein the electronic component has a thickness of 500 microns or less, optionally 400 microns or less, optionally 300 microns or less, optionally 200 microns or less, optionally 100 microns or less, optionally 50 microns or less.

The vessel, syringe barrel, vial or cartridge of any embodiment, wherein the vessel, syringe barrel, vial or cartridge is configured such that the electronic component can withstand sterilization, optionally sterilization by e-beam or gamma irradiation, optionally sterilization by Eto or VHP gas.

The vessel, syringe barrel, vial or cartridge of any embodiment, in which the electronic component comprises a temperature sensor, optionally in which the electronic component is a passive RFID temperature sensor.

The vessel, syringe barrel, vial or cartridge of any embodiment, in which the electronic component comprises a pressure sensor, optionally in which the electronic component is a passive RFID pressure sensor.

The vessel, syringe barrel, vial or cartridge of any embodiment, in which the electronic component contains information regarding a defined dispense rate or defined range of acceptable dispense rates.

The vessel, syringe barrel, vial or cartridge of any embodiment, in which the information regarding the defined dispense rate or defined range of acceptable dispense rates is configured to be read by a delivery device, optionally an auto-injector or injection pen.

The vessel, syringe barrel, vial or cartridge of any embodiment, in which the electronic component contains information regarding a defined needle insertion depth or defined range of acceptable needle insertion depths.

The vessel, syringe barrel, vial or cartridge of any embodiment, in which the information regarding the defined needle insertion depth or defined range of acceptable needle insertion depths is configured to be read by a delivery device, optionally an auto-injector or injection pen.

The vessel, syringe barrel, vial or cartridge of any embodiment, in which the electronic component is configured to register an interrogation event into a database.

The vessel, syringe barrel, vial or cartridge of any embodiment, in which the inner surface, the outer surface, or both of the wall in which the electronic component is embedded and enclosed are identical or substantially identical to those of the wall of the same vessel, syringe barrel, vial or cartridge prepared without the electronic component.

The vessel, syringe barrel, vial or cartridge of any embodiment, in which the inner surface, the outer surface, or both of the wall in which the electronic component is embedded and enclosed are free of surface irregularities, such as those caused by insertion of an electronic component.

The vessel, syringe barrel, vial or cartridge of any embodiment, in which the electronic component comprises information, optionally identification information, stored directly on the component.

The vessel, syringe barrel, vial or cartridge of any embodiment, in which the electronic component is readable without connection to a computing network such as a cloud-computing network.

The vessel, syringe barrel, vial or cartridge of any embodiment, in which the electronic component is writable without connection to a computing network such as a cloud-computing network.

Embodiments of the present disclosure are directed to methods of preparing a vessel having an electronic component embedded in a thermoplastic material that makes up at least a portion of the vessel, the method comprising inserting the electronic component in a mold, and injecting thermoplastic material into the mold to form the vessel or at least a portion of the vessel, wherein the thermoplastic material surrounds or substantially surrounds the electronic component, thereby embedding the electronic component within a portion of the vessel. In some embodiments, the vessel may be a syringe barrel, medical cartridge, or vial.

Embodiments of the present disclosure are directed to methods of preparing a vessel having an electronic component embedded in a thermoplastic material that makes up at least a portion of the vessel, the method comprising providing a preform having an electronic component embedded therein, optionally in which the electronic component is embedded in an upper portion of the preform that corresponds with a neck of the vessel, and injection blow molding or injection stretch blow molding the preform to produce the vessel. In some embodiments, the vessel may be a syringe barrel, medical cartridge, or vial.

Embodiments of the present disclosure are directed to methods of preparing a syringe barrel having an electronic component embedded in a thermoplastic material that makes up at least a portion of the syringe barrel, the method comprising inserting the electronic component in a mold, and injecting thermoplastic material into the mold to form the syringe barrel or at least a portion of the syringe barrel, wherein the thermoplastic material surrounds or substantially surrounds the electronic component, thereby embedding the electronic component within a portion of the syringe barrel.

Embodiments of the present disclosure are directed to methods of preparing a syringe barrel having an electronic component embedded in a thermoplastic material that makes up at least a portion of the syringe barrel, the method comprising injection molding a first polymeric layer; cooling the first polymeric layer, e.g. to a temperature below its glass transition temperature; positioning the electronic component on a surface of the first polymeric layer; and injection molding a second polymeric layer, such that the second polymeric layer covers the electronic component.

Embodiments of the present disclosure are directed to methods of preparing a vial having an electronic component embedded in a thermoplastic material that makes up at least a portion of the vial, the method comprising providing a preform having an electronic component embedded therein, optionally in which the electronic component is embedded in an upper portion of the preform that corresponds with the neck of the vial; and injection blow molding or injection stretch blow molding the preform to produce the vial.

The method of any embodiment, further comprising inspecting the vessel, syringe barrel, or vial to ensure that presence, positioning, and/or functioning of the electronic component.

The method of any embodiment, wherein the inspecting comprises an automated machine-operated visual inspection of the vessel, syringe barrel, or vial to identify the presence of the electronic component.

The method of any embodiment, wherein the machine-operated visual inspection also determines whether the electronic component is in an acceptable location within the vessel, syringe barrel, or vial.

The method of any embodiment, wherein the inspecting comprises using a remote reader to ensure that the electronic component is readable.

The method of any embodiment, wherein the electronic component is an RFID tag, optionally an RFID tag that utilizes one or more frequencies within the range of 865 to 928 MHz, optionally an RFID tag that utilizes a frequency of about 13.56 MHz.

The method any embodiment, wherein the electronic component comprises an integrated circuit.

The method of any embodiment, wherein the electronic component comprises data storage.

The method of any embodiment, wherein the thermoplastic material comprises one or more of the following: polypropylene, polyethylene, COP, COC, or CBC.

The method of any embodiment, wherein no part of the electronic component is on the exterior surface of the vessel, syringe barrel, vial or cartridge, and optionally wherein no part of the electronic component is on an interior surface of the vessel, syringe barrel, vial, or cartridge.

The method of any embodiment, wherein the electronic component is embedded in a hub portion of the syringe barrel.

The method of any embodiment, wherein the electronic component is embedded in a transition region between a main body of the syringe barrel and a hub portion of the syringe barrel.

The method of any embodiment, wherein the hub portion is a needle hub or a Luer hub.

The method of any embodiment, wherein the syringe barrel is configured so that the electronic component is visually concealed by a rigid needle shield or Luer cap.

The method of any embodiment, wherein the electronic component is embedded in a main body of the syringe barrel adjacent a rear flange.

The method of any embodiment, wherein the electronic component is embedded in a neck portion of the vial, optionally a thickened flange region.

The method of any embodiment, wherein the vial is configured so that the electronic component is visually concealed by a stopper and crimp closure.

The method of any embodiment, wherein the vessel is a cartridge and the electronic component is embedded in a needle attachment portion of the cartridge.

The method of any embodiment, wherein the cartridge is configured so that the electronic component is visually concealed by a cap placed on the needle attachment portion.

The method of any embodiment, wherein the vessel, syringe barrel, vial or cartridge further comprises a label and wherein the electronic component is embedded in a portion of the vessel, syringe barrel, vial or cartridge that is visually concealed by the label.

The method of any embodiment, wherein the cartridge is configured so that the electronic component is directly readable by a delivery device, optionally an auto-injector or an injection pen.

The method of any embodiment, wherein the vessel, syringe barrel, vial or cartridge is configured so that the electronic component is visually concealed by a portion of a finished pharmaceutical package.

The method of any embodiment, wherein the electronic component is detectable by automated visual inspection of the vessel, syringe barrel, vial or cartridge in an empty state.

The method of any embodiment, wherein the electronic component is configured to be written to using an external writer.

The method of any embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

The method of any embodiment, wherein the electronic component has a thickness of 500 microns or less, optionally 400 microns or less, optionally 300 microns or less, optionally 200 microns or less, optionally 100 microns or less, optionally 50 microns or less.

The method of any embodiment, wherein the vessel, syringe barrel, vial or cartridge is configured such that the electronic component can withstand sterilization, optionally sterilization by e-beam or gamma irradiation, optionally sterilization by Eto or VHP gas.

The method of any embodiment, in which the inner surface, the outer surface, or both of the wall of the vessel, syringe barrel, vial or cartridge in which the electronic component is embedded and enclosed are identical or substantially identical to those of the wall of the same vessel, syringe barrel, vial or cartridge prepared without the electronic component.

The method of any embodiment, in which the inner surface, the outer surface, or both of the wall of the vessel, syringe barrel, vial or cartridge in which the electronic component is embedded and enclosed are free of surface irregularities, such as those caused by insertion of an electronic component.

The method of any embodiment, in which the step of providing a preform having an electronic component embedded therein comprises injection molding a first polymeric layer; cooling the first polymeric layer, e.g. to a temperature below its glass transition temperature; positioning the electronic component on a surface of the first polymeric layer; and injection molding a second polymeric layer, such that the second polymeric layer covers the electronic component.

The method of any embodiment, in which the step of providing a preform having an electronic component embedded therein further comprises transferring a polymeric body resulting from cooling of the first polymer layer from a first injection mold to a second injection mold; and injection molding the second polymeric layer in the second injection mold.

The method of any embodiment, in which the electronic component is positioned at a portion of the preform so that the step of injection blow molding or injection stretch blow molding to produce the vessel or vial places substantially no stresses on the electronic component.

The method of any embodiment, in which the method of preparing a syringe barrel having an electronic component embedded in a thermoplastic material that makes up at least a portion of the syringe barrel further comprises transferring a polymeric body resulting from cooling of the first polymer layer from a first injection mold to a second injection mold; and injection molding the second polymeric layer in the second injection mold.

The method of any embodiment, wherein the syringe barrel resulting from the molding process comprises a needle, the proximal end of which is embedded in the needle hub.

The method of any embodiment, wherein the injection molding of the first polymer layer, the positioning of the electronic component, and the injection molding of the second polymer layer are performed without moving the needle or mold core relative to one another.

The method of any embodiment, wherein a portion of an injection mold surrounding the needle hub opens to enable the positioning of the electronic component.

The method of any embodiment, wherein the needle is securely embedded a polymeric body resulting from cooling of the first polymer layer.

The method of any embodiment, wherein the needle is inserted into the second injection mold, optionally in which the needle is not present in the first injection mold.

The method any embodiment, in which the electronic component comprises information, optionally identification information, stored directly on the component.

The method of any embodiment, in which the electronic component is readable without connection to a computing network such as a cloud-computing network.

The method of any embodiment, in which the electronic component is writable without connection to a computing network such as a cloud-computing network.

Embodiments of the present disclosure are directed to methods of monitoring whether or not a pharmaceutical package of any embodiment is maintained within defined storage conditions, comprising providing a pharmaceutical package according to any embodiment; registering any non-compliance events detected by the temperature sensor (if applicable), the pressure sensor (if applicable), or both.

Embodiments of the present disclosure are directed to methods of facilitating administration of the pharmaceutical solution contained within the lumen of a pharmaceutical package according to any embodiment comprising interrogating the electronic component prior to administration, thereby obtaining information regarding whether there has been a non-compliance event detected by the temperature sensor (if applicable), the pressure sensor (if applicable), or both, and optionally rejecting the pharmaceutical package if a non-compliance event was detected.

The method of any embodiment, wherein the interrogating is performed by a drug delivery device, optionally an auto-injector or injection pen.

The method of any embodiment, wherein the drug delivery device is configured to reject the pharmaceutical package if a non-compliance event is detected.

Embodiments of the present disclosure are directed to methods of facilitating proper administration of the pharmaceutical solution contained within the lumen of a pharmaceutical package according to any embodiment, comprising: interrogating the electronic component prior to administration, thereby obtaining the information regarding one or more administration parameters; and administering the pharmaceutical solution in accordance with the one or more administration parameters.

The method of any embodiment, wherein the interrogating and administering is performed by a drug delivery device, optionally an auto-injector or injection pen.

The method of any embodiment, further comprising adjusting one or more settings of the drug delivery device to comply with the one or more administration parameters, optionally wherein the adjusting is performed automatically by the drug delivery device.

Embodiments of the present disclosure are directed to methods of monitoring administration of pharmaceutical packages, e.g. as part of a clinical trial, comprising providing pharmaceutical packages according to any embodiment; registering, to a database, the date and time of interrogations of the electronic components, and/or non-compliance events detected by the temperature sensor (if applicable), the pressure sensor (if applicable), or both.

Embodiments of the present disclosure are directed to methods of providing a pharmaceutical product with identification information, comprising: obtaining a vessel, syringe barrel, vial or cartridge according to any of the preceding claims, optionally wherein the electronic component comprises identification information relating to the vessel, syringe barrel, vial or cartridge; filling the vessel with a pharmaceutical formulation; and writing information relating to the pharmaceutical formulation to the electronic component, optionally without connection to a computing network such as a cloud-computing network.

The method of any embodiment, wherein the identification information relating to the vessel, syringe barrel, vial or cartridge is readable directly from the electronic component.

### BRIEF DESCRIPTION OF THE DRAWINGS

FIG. 1A is a cross-sectional view of an embodiment of a staked needle syringe barrel having an electronic component embedded in and fully enclosed by the needle hub portion of the syringe barrel.

FIG. 1B is a cross-sectional view of (a) an embodiment of a pre-filled syringe having an electronic component embedded in and fully enclosed by the needle hub portion of the syringe barrel and concealed by a conventional rigid needle shield and (b) an embodiment of a pre-filled syringe having an electronic component embedded in and fully enclosed by the Luer hub portion of the syringe barrel and concealed by a conventional Luer cap.

FIG. 2 is a partial cross-sectional view of an embodiment of a pre-filled, staked needle syringe, showing an electronic component embedded in and fully enclosed by the needle hub portion of the syringe barrel and concealed by a conventional rigid needle shield.

FIG. 3 is a reproduction of an image demonstrating that automated visual inspection is capable of detecting an electronic component embedded in and fully enclosed by a hub portion of a syringe barrel in accordance with embodiments of the present disclosure.

FIG. 4A is a cross-section view of an embodiment of a vial having an electronic component embedded in and fully enclosed by the neck portion, and more particularly the thickened flange region of the neck portion, of the vial.

FIG. 4B is a cross-sectional view of an embodiment of a pharmaceutical vial having an electronic component embedded in and fully enclosed by the neck portion, and more particularly the thickened flange region of the neck portion, of the vial and concealed by a conventional closure assembly.

FIG. 5 is a schematic showing a method of incorporating an electronic component into a vial in accordance with an embodiment of the present disclosure.

FIG. 6 is a perspective view of a set of vials having electronic components embedded in and fully enclosed by the neck portion of the vial and concealed by a conventional closure assembly in accordance with an embodiment of the present disclosure.

### DETAILED DESCRIPTION

The present invention will now be described more fully, with reference to the accompanying drawings, in which several embodiments are shown. This invention can, however, be embodied in many different forms and should not be construed as limited to the embodiments set forth here. Rather, these embodiments are examples of the invention, which has the full scope indicated by the language of the claims. Like numbers refer to like or corresponding elements throughout. The following disclosure relates to all embodiments unless specifically limited to a certain embodiment.

The electronic component can be selected from among known electronic components, including for example a RFID tag or an integrated circuit (IC). Where an RFID component is utilized, the frequency or frequencies of transmission may fall within the range of 865 to 928 MHz, or a subset of that range such as 902-928 MHz, 865.5-867.5 MHz, or about 915 MHz. In other embodiments where an RFID component is utilized, the frequency of transmission may be about 13.56 MHz.

By embedding an electronic component in the vessel and the resulting pharmaceutical packaging, the component may contain information that can be read and optionally modified through a wireless interface. This will enable enhanced inventory control and security through improved identification, routing, and tracking of products. Because the electronic component may be embedded within a wall of the vessel itself (and not located on the exterior surface of the vessel), the electronic component may be protected from damage, discretely incorporated and cannot be separated physically from the packaging, which are major improvements over previously suggested tags. Further, molding the electronic component directly into the wall of the container allows for lost-cost and repeatable production without the need for significant additional manufacturing steps or specially designed or customized components. In some embodiments, for instance, vials, syringes, cartridges, and the like may be provided with an electronic component with no change to the materials and/or dimensions of the existing vial, syringe, or cartridge designs. In some embodiments, the electronic component may also be attached to or incorporated into the vessel, e.g. molded into a portion of a thermoplastic vessel, in a fully automated manner. Optional process conditions for the molding of vials, syringes, and cartridges are disclosed in the following, each of which is incorporated herein by reference in its entirety: U.S. Pat. Nos. 9,381,687; 10,639,421; 9,475,225; and International Application No. PCT/US2019/061293 (published as WO2020102434A2).

In some embodiments, the electronic component may be positioned on the vessel/package so that the electronic component is not visible to downstream users such as pharmacies and healthcare professionals. For example, in some embodiments, the electronic component may be embedded into the neck portion of a vial, which may subsequently be concealed by the closure, e.g. stopper and metal crimp. Similarly, the electronic component may be embedded into the needle hub portion of a syringe barrel, or the transition region between the primary barrel portion and the hub portion of a syringe barrel, which may subsequently be concealed by a rigid needle shield (or in the case of Luer lock syringe, a cap). Because the electronic component is not visible and does not require significant alterations to existing vessel designs, it will have no impact on existing processes and quality checks used within the pharmaceutical industry and throughout the supply chain of the pharmaceutical package.

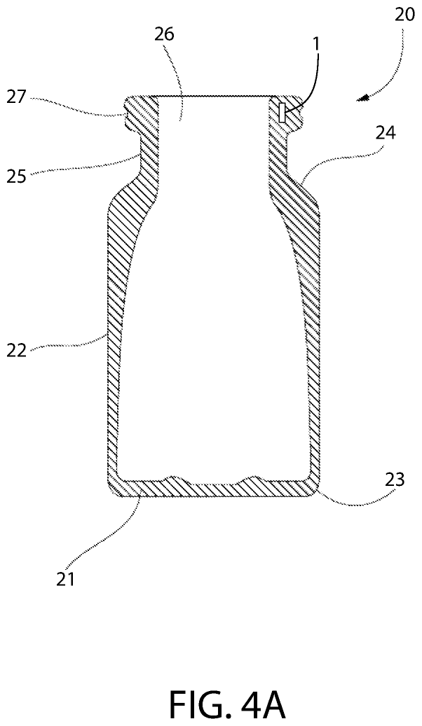

Further, each vessel may be inspected to confirm the presence and/or functioning of the electronic component prior to being filled and sealed, thereby preventing filled vessels from having to be disposed due to problems with the electronic component or that may arise due to attachment of the electronic component post-filling. The inspection may, in full or in part, be performed visually, e.g. using a high resolution camera of the sort that are already routinely used for quality control of the vessels, to detect the presence and location of the electronic component. An example image from an automated, machine-based visual inspection of the sort that could identify an electronic component 1 embedded in a hub portion 12 of a syringe barrel is shown in FIG. 3. The inspection may also, in full or in part, be performed electronically, e.g. by ensuring that the electronic component can be detected by an appropriate reader.

In some embodiments, the electronic component may be configured to enable connected drug delivery options. For instance, in some embodiments, a syringe or cartridge may be provided with an electronic component, e.g. RFID tag, as described herein and an auto-injector, pen, or other delivery system may be provided with a reader that is configured to read the electronic component, e.g. RFID tag, directly in order to ensure that the pharmaceutical package is suitable for administration. In other embodiments, a pharmaceutical package such as a vial, syringe, or cartridge may be provided with an electronic component, e.g. RFID tag, as described herein and an administrator may use a reader, including for instance an independent reader or one associated with a smartphone or tablet, to read the electronic component, e.g. RFID tag, in order to ensure that the pharmaceutical package is suitable for administration.

For instance, where certain drugs must be maintained within a defined temperature range, the electronic component, e.g. RFID tag, may monitor the temperature of the pharmaceutical package, e.g. through an associated temperature sensor such as the passive RFID temperature sensors described in Virtanen et al., Temperature Sensor Tag for Passive UHF RFID Systems, published in 2011 IEEE Sensors Applications Symposium, 22-24 Feb. 2011, and available here: https://ieeexplore.ieee.org/document/5739788, the entirety of which is incorporated herein by reference, and a reader may be used to interrogate the electronic component in order to ensure that the package and/or drug formulation has been maintained within the defined temperature range before administration. Similarly, where certain drugs must be maintained within a defined pressure range, the electronic component, e.g. RFID tag, may monitor the pressure of the pharmaceutical package, e.g. through an associated pressure sensor such as the passive RFID pressure sensors manufactured by Farsens under the names Vortex and Cyclon, and a reader may be used to interrogate the electronic component in order to ensure that the package and/or drug formulation has been maintained within the defined pressure range before administration.

In some embodiments, the electronic component, e.g. RFID tag, may also be configured to facilitate and/or ensure proper administration of the fluid contained within the pharmaceutical package. For instance, in some embodiments, a pharmaceutical package such as a vial, syringe, or cartridge, may be provided with an electronic component as described herein that contains information regarding proper administration of the fluid, e.g. one or more delivery parameters. That information may be obtained from the electronic component, e.g. by a reader, and a user/administrator provided with guidance regarding delivery parameters by which he/she can ensure that those delivery parameters are satisfied. Further, in some embodiments, a pharmaceutical package such as a vial, syringe, or cartridge, may be provided with an electronic component that contains information regarding proper administration of the fluid, e.g. one or more delivery parameters, and an auto-injector, pen, or other delivery system may be provided with a reader that is configured to read the electronic component, e.g. RFID tag, and then control certain delivery parameters and/or ensure that certain delivery parameters identified from the electronic component are satisfied.

In some embodiments, the electronic component, e.g. RFID tag, may contain information relating to the strength of the pharmaceutical formulation contained in the lumen of the vessel. For drugs that may be provided at multiple different strengths (e.g. concentration of active agent, total amount of active agent, etc.), it is important to ensure that the proper dosage is administered to a patient. By providing information relating to the strength of the pharmaceutical formulation on the electronic component, e.g. RFID tag, an administrator of the formulation may obtain the strength of the pharmaceutical formulation by reading the electronic component. In some embodiments, the accessing of this information may be recorded, e.g. to a data log, and used to ensure that administrators are performing the necessary checks (or double-checks, as the information may also be provided on a label) of the strengths of pharmaceutical products before administration.

Some drugs, and in particular those for cancer treatments and autoimmune disease treatments as well as mRNA drugs, and the like require that the drug formulation be dispensed at a defined dispense rate or a dispense rate within a defined range, at a defined needle insertion depth or a needle insertion depth within a defined range (e.g. to a particular region such as subcutaneous, intra-muscular, and the like), a combination thereof, or the like.

In some embodiments, the electronic component, e.g. RFID tag, may contain information regarding a defined drug dispense rate or a defined range of acceptable drug dispense rates. A delivery system, e.g. auto-injector, pen, or other delivery system, may be configured to read the electronic component and automatically set one or more injection parameters associated with control of the drug dispense rate, e.g. the force applied to the plunger, so as to ensure that the dispense rate of the drug formulation meets the defined rate or falls within the defined acceptable range. In other embodiments, an administrator may obtain the information from the RFID tag, e.g. byway of a reader such as an independent reader or one that may be associated with a smartphone, tablet computer, or other device, and either manually set one or more injection parameters of a delivery device or manually control the dispense rate to ensure that it meets the defined dispense rate or falls within the defined range.

Further, in some embodiments, either the electronic component, e.g. RFID tag, or the delivery system, e.g. auto-injector, may be configured to detect the speed by which the plunger is advanced forward within the syringe barrel, which corresponds with the drug dispense rate. The delivery system may respond to a reading that does not correlate with the defined dispense rate or falls outside of the defined range of acceptable dispense rates—obtained from the electronic component, e.g. RFID tag—by automatically adjusting one or more injection parameters, e.g. the force applied to the plunger, in order to adjust the dispense rate upward or downward as necessary to meet the defined dispense rate or fall within the defined acceptable range. Alternatively, the delivery system may alert the administrator that the dispense rate is too high or too low so that the administrator may manually adjust the dispense rate upward or downward as necessary to meet the defined dispense rate or fall within the defined acceptable range. In some embodiments, for instance, the delivery device may comprise one or more tachometers, accelerometers, combinations thereof, and the like.

In some embodiments, the electronic component, e.g. RFID tag, may contain information regarding a defined needle insertion depth or range of acceptable needle insertion depths. A delivery system, e.g. auto-injector, pen, or other delivery system, may be configured to read the electronic component and to set one or more injection parameters associated with control of the needle insertion depth, e.g. the force applied to the syringe barrel during needle insertion, so as to ensure that the insertion depth of the needle meets the defined depth or falls within the defined acceptable range. In other embodiments, an administrator may obtain the information from the RFID tag, e.g. byway of a reader such as an independent reader or one that may be associated with a smartphone, tablet computer, or other device, and either manually set one or more injection parameters of a delivery device or control the needle insertion depth manually to ensure that it meets the defined depth or falls within the defined range.

Further, in some embodiments, either the electronic component, e.g. RFID tag, or the delivery device may be configured to detect the proximity of the injection site, which corresponds with the needle insertion depth. The delivery system may respond to a reading that does not correlate with the defined needle insertion depth or falls outside of the defined range of acceptable needle insertion depths—obtained from the electronic component, e.g. RFID tag—by automatically adjusting one or more parameters in order to adjust the needle insertion depth as necessary to meet the defined needle insertion depth or fall within the defined acceptable range. Alternatively, the delivery system may alert the administrator that the needle insertion depth is too high or too low so that the administrator may manually adjust the needle insertion depth as necessary to meet the defined needle insertion depth or fall within the defined acceptable range. In some embodiments, for instance, the delivery system may comprise a proximity sensor, directed axially forward (parallel to the needle) and configured to measure the proximity of the injection site, which relates to the needle insertion depth, during needle insertion.

In some embodiments, the electronic component, e.g. RFID tag, may be used by an administrator to enable record collection. For instance, many medications may need to be administered to patients in a home or group home setting. Often, a health care professional will have to administer medications to multiple patients at a group home or across a number of homes and the record keeping for those administrations may be complicated and time-consuming. The integrated RFID tag of embodiments of the present disclosure may simply such record collection, e.g. by recording a day/time, location, or both of a reading and storing that information in a data log.

In some embodiments, the electronic component may comprise a storage element and may itself be used for data storage. In other words, the container may itself serve as the data carrier, rather than the cloud. The data may also easily be transferred from the container, i.e. from the electronic component, to a data cloud. The electronic component is configured to be interrogated by an external reader and, optionally written to using an external writer.

Embodiments of pharmaceutical packages, e.g. syringes, cartridges, and vials, as described herein may provide any combination of the above capabilities, thereby improving patient or administrator adherence to the delivery guidelines for a specific drug formulation.

Moreover, in some embodiments, the RFID tag may be loaded with particular information relating to the identity, storage conditions, and/or administration parameters, of an emerging drug, in order to enhance the effectiveness and reliability of a clinical trial. For many emerging drugs, the effectiveness of clinical trials is dependent on the maintenance of certain protocols relating for example to the temperature of the drug, the depth of the injection, and/or the rate of injection into the patient. The use of an RFID tag associated with a pharmaceutical package as described above could be used to ensure proper compliance with those parameters. Embodiments of the present disclosure are directed to methods, e.g. of performing clinical trials, that include providing pharmaceutical packages having RFID tags attached or incorporated therein, and prompting or otherwise causing one to interrogate the RFID tag of a pharmaceutical package to determine whether one or more protocols has been maintained and/or to determine certain protocols associated with delivery of the pharmaceutical formulation contained therein prior to administration of the pharmaceutical formulation.

The RFID tags may also be used to demonstrate, e.g. for post approval studies, that the drug has been administered in a manner consistent with the controlled protocols of a clinical study. In some embodiments, for instance, the electronic component, e.g. RFID tag, may be configured to log certain data, e.g. drug storage data, drug administration data, etc., in a central database. For instance, when interrogated by a reader prior to administration, as described herein, the RFID tag may register that interrogation (corresponding with the date/time of administration) into a central data log. Having every package, e.g. vial, syringe, or cartridge, utilized in the clinical trial register to the same data log would thereby produce a reviewable record of all administrations during a clinical trial. The RFID tag may also be configured to register any data which is inconsistent with clinical trial protocols, e.g. any deviations from a defined temperature storage protocol or the like, into the data log. That sort of monitoring may help produce a reviewable record of the clinical trial and ensure that post approval studies and surveillance of drug effectiveness in a larger patient population is properly evaluated.

Embodiments of the present disclosure are directed to methods, e.g. of performing clinical trials, that include providing pharmaceutical packages having RFID tags attached or incorporated therein; optionally prompting or otherwise causing one to interrogate the RFID tag of a pharmaceutical package to determine whether one or more protocols has been maintained and/or to determine certain protocols associated with delivery of the pharmaceutical formulation contained therein prior to administration of the pharmaceutical formulation; optionally using the RFID tag to log the location, date, and/or time of that interrogation; and optionally using the RFID tag to log data associated with the clinical trial, e.g. drug storage data, into a central data log.

In some embodiments, telematics may be used. Telematics involves the technology of sending, receiving, and storing information using telecommunication devices to control remote objects. In the context of the present disclosure, the location where the pharmaceutical package is being administered, i.e. the location where the electronic component is interrogated prior to administration, can be used to access the relevant information from the clinical study. For example, if the pharmaceutical package was being administered in a specific region in Asia, then the appropriate clinical studies from that demographic population could be accessed.

In some embodiments, any combination of the data described herein may be stored on the electronic component, e.g. RFID tag, itself, as opposed to on a cloud-computing network. By having the data written directly to the electronic component, and thus directly to the vessel itself, embodiments of the present disclosure provide a number of benefits over embodiments in which the data is stored in a cloud. For instance, each vessel may be tracked by identification information stored directly on the electronic component, e.g. RFID tag, throughout the supply chain of a pharmaceutical product. This allows the identification information (e.g. unique identifier, batch number, pharmaceutical material number, etc.) to be consistent without having to connect the systems of multiple different entities (e.g. vessel manufacturer, filler, secondary packaging provider, shipper, etc.) to the same cloud network.

In some embodiments, storing data directly on the electronic component, e.g. RFID tag, also allows for the writing of data directly onto the electronic component at multiple steps across the supply chain. For instance, the vessel manufacturer may write vessel identification information to the electronic component; the filler may write information relating to the pharmaceutical composition that is filled into the lumen of the vessel, e.g. pharmaceutical material identification information, to the electronic component; a secondary packaging provider may optionally write additional identification information to the electronic component; etc. Notably, by providing a vessel having an integrated RFID tag on which data is stored directly, rather than in a cloud computing network, each different entity across the supply chain may add information to the RFID tag without the plurality of entities having to be connected to the same cloud network.

By allowing for the reading and/or writing of identification information directly from the electronic component, e.g. RFID tag, a vessel and its pharmaceutical contents may be tracked from the point of manufacture of the vessel to the point of administration of a pharmaceutical product without needing to connect a plurality of entities to a common cloud. This produces significant benefits in terms of cost savings and greatly simplifies supply chain management. For instance, there is no need for independent entities to obtain equipment and/or build out the infrastructure to connect to a shared cloud, to share data over a shared cloud (which may often be undesired), etc.

An electronic component may be attached to the vessel and/or pharmaceutical package through any of a variety of methods: adhesive, mechanical attachment or over molding. As described herein, a molding technique where the electronic component becomes part of the package during the molding process, and preferably one in which the electronic component is embedded in and fully enclosed by a wall of the vessel, is desirable, as it provides a number of benefits over the other methods.

In some embodiments, for instance, the electronic component may be embedded into a portion of a thermoplastic vessel by mold labeling. Using this technique, the electronic component (also referred to as an inlay) may be laminated with a film substrate. The film substrate is desirably made from a plastic that is compatible with the pharmaceutical package. In a mold labeling process, the inlay is inserted into the mold prior to plastic injection. In some embodiments, for instance, the inlay may be presented to the mold in a label format. During the injection phase of the vessel molding process, the inlay is overmolded and the inlay becomes an integral part of the package.

Some of the materials used to manufacture thermoplastic vessels used as pharmaceutical packages include but are not limited to: polypropylene, polyethylene, a cyclic olefin polymer (COP), a cyclic olefin copolymer (COC), and a cyclic block copolymer (CBC).

Embodiments of the present disclosure are directed to pharmaceutical vials 20 made of plastic and having an electronic inlay 1 incorporated into the plastic wall of the vial. More particularly, embodiments of the present disclosure are directed to pharmaceutical vials 20 made of plastic and having an electronic inlay 1 embedded in and fully enclosed by the plastic wall of the vial.

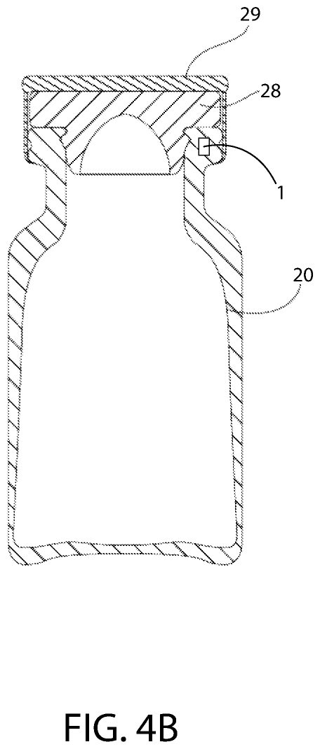

Vials 20 of the present disclosure may include a bottom wall 21, a side wall 22 extending upward from the bottom wall, a curved lower edge joining the bottom wall and the side wall 23, a radially inwardly extending shoulder 24 formed at the top of the side wall, and a neck 25 extending upwardly from the shoulder, the neck defining an opening 26 at the top thereof, the opening leading to the vial interior, i.e. lumen. In many embodiments, the neck may comprise an outwardly extending flange 27 which is configured to receive part of a closure. An example of such a vial 20 is shown in FIG. 4A.

Once filled with a pharmaceutical product, typically an injectable pharmaceutical product, the vial 20 is sealed. Typically a pharmaceutical vial 20 is sealed with a two-part closure comprising a rubber stopper 28 and a cap 29, such as an crimp cap, a Flip-Off® Seal of the sort produced by West Pharmaceutical, or the like. A crimp cap is typically made of a metal such as aluminium and is crimped over the top of the stopper 28 and the neck flange 27 of the vial. The combination of filled vial 20, stopper 28, and cap 29, may be referred to as a pharmaceutical vial or vial package. An example of a pharmaceutical vial package is shown in FIG. 4B.

In some embodiments, the electronic inlay 1 may be embedded in and fully enclosed by the plastic that makes up the neck 25 of the vial, for example in the thickened plastic region that makes up the upper flange 27.

The upper portion of the neck 25, including the upper flange 27, must be prepared with relatively low dimensional tolerances in order to ensure consistent sealing with commercial stoppers 28 and caps 29. Accordingly, both the inner surface of the neck portion 25 and the outer surface of the neck portion, including flange 27, must have uniform surfaces. In some embodiments, the upper flange 27 may also have one or more exterior surface features, which may assist with gripping and/or with securing of a cap 29.

Pharmaceutical vials 20 that are made of plastic are prepared by blow molding, e.g. using an injection blow molding or injection stretch blow molding technique. An example of an injection stretch blow molding technique is shown in FIG. 4. In a first step 41, a preform 30 is injection molded. A thermoplastic resin 33, for example COP or COC resin, is melted and delivered to an injection mold where it is formed into the shape of a preform 30. The preform 30 is then injection stretch blow molded to form the vial 20. In a second step 42 of such a process, the preform 30 is heated above its glass transition temperature and stretched, e.g. by a mandrel 34 or core rod of a stretch blow molding machine. In a third step 43 of such a process, high-pressure air is blown into the preform 30, thereby expanding the preform until it contacts the mold and thus takes the shape of a vial 20. The stretching 42 may be performed in advance of the blow molding 43 as shown in FIG. 4, or as part of the blow molding 43, e.g. through a core rod of a stretch blow molding machine (in which case the process may be viewed as a two-step process). In other embodiments, e.g. when the vial 20 is prepared by extrusion blow molding, the stretching step 42 may be omitted altogether.

During the blow molding process, the neck portion 31 of the preform 30 is held in place and, unlike the rest of the preform, does not undergo deformation. Accordingly, the neck portion 31 of the preform 30 is injection molded to have the dimensions and elements, e.g. the flange 27 and any surface features of the flange, of the neck portion 25 of the resulting pharmaceutical vial 20.

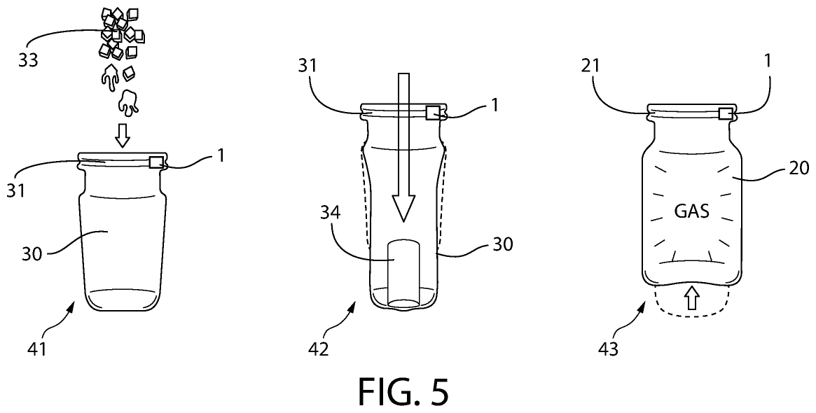

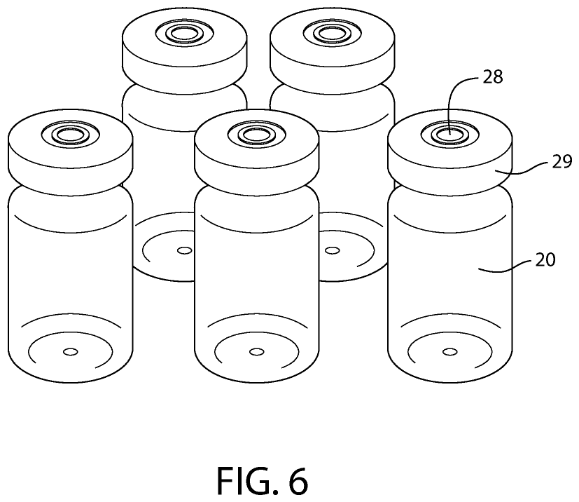

To incorporate the electronic inlay 1 into the vial 20, the inlay may be incorporated during the step, e.g. step 41, of injection molding a preform 30. In particular, the electronic inlay 1 may be incorporated in the upper portion 31 of the preform 30 (corresponding with the neck region 25 of the vial) that is not dimensionally affected by the downstream blowing and optional stretching steps. This allows the electronic inlay 1 to avoid being subjected to the stresses that would result if the inlay were positioned in a portion of the preform that was converted into the sidewall 22 or base 21 of the vial during the blow-molding process. It also has the additional benefit of allowing the electronic component 1 to be concealed by the closure assembly 28, 29 that is subsequently applied to the filled vial, as shown for example in FIG. 5.

Desirably, the electronic inlay 1 is embedded and fully enclosed by the plastic wall of the vial 20. In that way, the inner and outer surfaces of the vial 20 may be identical or substantially identical (i.e., made of the same polymer material and lacking any surface irregularities resulting from insertion of the electronic inlay) as those of a vial prepared without the electronic inlay 1. In some embodiments, the electronic inlay 1 is embedded and fully enclosed by the plastic that makes up the neck portion 25 of the vial, and more particularly in the thickened flange region 27.

Desirably, the electronic inlay 1 is embedded and fully enclosed by the plastic wall of the vial in a manner that does not cause surface irregularities or variability. For instance, the electronic inlay 1 may be introduced into the neck portion 31 of the preform during the step 41 of injection molding the preform 30. In such a process, the injection molding of the preform 30 would be performed in multiple steps, similar to a two-shot molding process or an overmolding process. In a first step, a first amount of the thermoplastic material 33, for example COP or COC resin, is melted and delivered to an injection mold where it forms a first layer of polymer. The electronic inlay 1 is then positioned at a desired location on the surface of the first layer. This may involve cooling of the first layer of polymer in advance of placing the electronic inlay 1 on its surface, particularly where the overall thickness of the first layer is small. After the electronic inlay 1 is positioned on the surface of the first layer, a second injection of the thermoplastic material 33 is delivered to the injection mold, where it flows over top of the surface of the first layer and the electronic inlay positioned thereon. The molded preform 30 is then cooled and removed from the mold. In that manner, the electronic inlay 1 may be sandwiched between the first and second polymer layers, with the result being a preform wall having the electronic inlay fully embedded and enclosed therein.

In some embodiments, the injection molding step 41 may involve the use of two injection molds. In a first step, a first amount of the thermoplastic material 33, for example COP or COC resin, is melted and delivered to a first injection mold where it forms an intermediate polymeric body. The intermediate polymeric body is then cooled and transferred to a second injection mold. The electronic inlay 1 is positioned at a desired location on the surface of the intermediate polymeric body, optionally after the intermediate polymeric body is placed within the second injection mold or during transfer of the intermediate polymeric body to the second injection mold. A second amount of the thermoplastic material 33, for example COP or COC resin, is melted and delivered to the second injection mold, where it flows over top of the surface of the intermediate polymeric body and the electronic inlay 1 positioned thereon to form a final molded preform 30. The final molded preform 30 is then cooled and removed from the second injection mold. As described above, the result is a preform wall having the electronic inlay fully embedded and enclosed therein.

Where the preform 30 contains the electronic inlay 1 in the upper portion 31 of the preform that is injection molded to correspond with the neck 25 of the vial, and optionally with the thickened flange portion 27 of the neck, the preform may then be converted into a vial 20 by a conventional blow molding process, such as that described above. Because the electronic inlay 1 is embedded and fully enclosed by the upper portion 31 of the preform that is not subjected to deformation during the blow molding process, the electronic inlay is not subjected to any real stresses during the blow molding process and its presence does not interfere with the blow molding and shaping of the vial 20.

Moreover, by incorporating the electronic inlay 1 into the vial 20 in this manner, the dimensions of the necks 25 of the vials, and optionally the thickened flange portions 27 of the necks, may be repeatably produced within tight tolerances. Further, since the electronic inlay 1 is not introduced into the preform 30 or vial 20 during or after the blow molding process, the exterior and interior surfaces of the vial walls in which the electronic inlays are embedded and enclosed may be uniform. In some embodiments, for instance, both the interior surface of the vial neck 25 and the exterior surface of the vial neck may be free of surface irregularities cause by electronic inlay insertion. For instance, the interior surface of the vial neck 25 may be smooth and the exterior surface may either be smooth or may contain one or more conventional surface features, e.g. such as that shown in FIGS. 4A and 4B. Finally, because the electronic inlay 1 is fully enclosed by the polymeric wall of the vial 20 and thus no portion of the electronic inlay is exposed on either the interior or exterior surfaces of the vial.

Though incorporation of an electronic inlay 1 into the neck portion 25 of a vial 20 is complicated by the fact that the neck portions of a plurality of vials must be produced with uniform surfaces and tight dimensional tolerances (to enable effective sealing with a closure assembly 28, 29), the above-described process allows for the production of vials having such uniform surfaces and tight dimensional tolerances.

Embodiments of the present disclosure are directed to pre-filled syringes 10 made of plastic and having an electronic inlay 1 incorporated into the plastic wall of the syringe barrel. More particularly, embodiments of the present disclosure are directed to syringe barrels made of plastic and having an electronic inlay 1 embedded in and fully enclosed by the needle or Luer lock hub 12 or in the transition region 13 between the primary portion of the syringe barrel 11 and the needle or Luer hub, and pre-filled syringes 10 that utilize such syringe barrels. In other embodiments, the syringe barrel may have an electronic inlay 1 embedded in and fully enclosed by the syringe barrel wall directly above the rear flange 14.

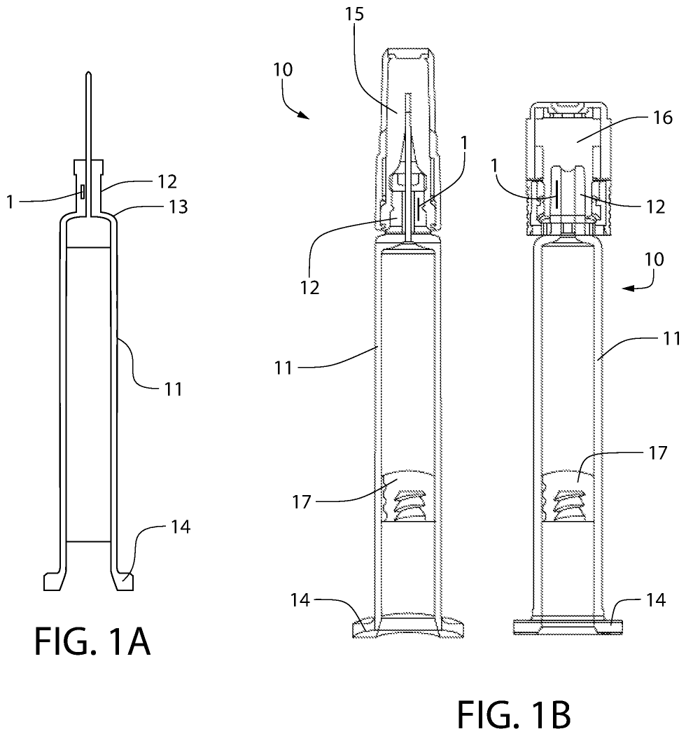

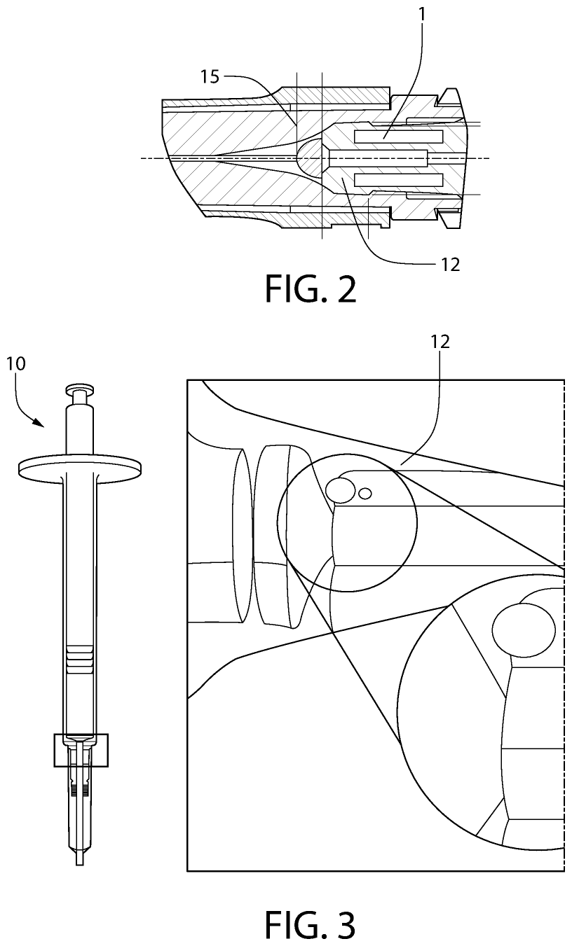

A pre-filled syringe typically comprises the syringe barrel, an injectable pharmaceutical composition contained within the lumen of the syringe barrel, a plunger 17 inserted into the rear opening of the syringe barrel to provide a rear seal, and either a rigid needle shield 15 or a Luer cap 16, depending on whether the syringe is a staked needle syringe or a Luer lock syringe. In some embodiments, a plunger rod may also be inserted into the rear opening of the syringe barrel and into communication with the plunger 17. In the case of a pre-filled syringe 10, the electronic inlay 1 may desirably be positioned in the needle or Luer lock hub 12 or in the transition region 13 between the primary portion of the syringe barrel 11 and the needle or Luer hub, as shown for example in FIG. 1. The positioning of the electronic inlay 1 in this manner has the advantage of allowing the electronic component to be concealed by the rigid needle shield 15 or Luer cap 16 that is subsequently applied to the pre-filled syringe, as shown for example in FIG. 1B. In alternative embodiments, the inlay 1 may be incorporated into the primary portion of a syringe barrel 11 wall, for example directly above the rear flange 14.

Desirably, the electronic inlay 1 is embedded and fully enclosed by the plastic that makes up either the wall of the syringe barrel 11, the transition region 13, or the hub portion 12. In that way, the inner and outer surfaces of the syringe barrel, and in particular the portion of the syringe barrel in which the electronic inlay 1 is embedded, may be identical or substantially identical (i.e., made of the same polymer material) as those of a syringe barrel prepared without the electronic inlay 1. In some embodiments, the electronic inlay 1 is embedded and fully enclosed by the plastic that makes up the needle or Luer hub 12 of the syringe barrel.

Desirably, the electronic inlay 1 is embedded and fully enclosed by the plastic wall of the vial in a manner that does not cause surface irregularities or variability. For instance, the electronic inlay 1 may be introduced into a portion of the syringe barrel as an intermediate step during molding. Plastic syringe barrels are typically produced by an injection molding process. In such a process, thermoplastic material 33, for example COP or COC resin, is melted and delivered to an injection mold where it forms the shape of the syringe barrel. For a staked needle syringe, the needle is held against a mold core during the injection molding process and the thermoplastic material flows around the sides of a proximal portion of the needle, thereby forming the needle hub portion 12 of the syringe barrel. When the thermoplastic material making up the needle hub portion 12 cools, the proximal portion of the needle is secured within the hub portion of the syringe barrel. In contrast, for a Luer lock syringe, one or more mold elements are used to form the flow passage that runs centrally through the hub portion 12.

In embodiments of the present disclosure, the electronic inlay 1 may be embedded into and fully enclosed by a portion of the syringe barrel, for instance the needle or Luer hub 12. To obtain such an arrangement, the injection molding of the syringe barrel may be performed in multiple steps, similar to a two-shot molding process or an overmolding process. In a first step, a first amount of the thermoplastic material 33, for example COP or COC resin, is melted and delivered to an injection mold where it forms a first layer of polymer. The first layer of polymer is then cooled to provide the first layer with a degree of rigidity, and the electronic inlay 1 is then positioned at a desired location on the surface of the first layer. After the electronic inlay 1 is positioned on the surface of the first layer, a second injection of the thermoplastic material 33 is delivered to the injection mold, where it flows over top of the surface of the first layer and the electronic inlay positioned thereon. The molded syringe barrel is then cooled and removed from the mold. In that manner, the electronic inlay 1 may be sandwiched between the first and second polymer layers, with the result being a syringe barrel having the electronic inlay fully embedded and enclosed within a portion of the wall, e.g. wall 11, transition region 13, flange 14, or hub portion 12.

In some embodiments, the injection molding may involve the use of two injection molds. In a first step, a first amount of the thermoplastic material 33, for example COP or COC resin, is melted and delivered to a first injection mold where it forms an intermediate polymeric body. The intermediate polymeric body is then cooled and transferred to a second injection mold. The electronic inlay 1 is positioned at a desired location on the surface of the intermediate polymeric body, optionally after the intermediate polymeric body is placed within the second injection mold or during transfer of the intermediate polymeric body to the second injection mold. A second amount of the thermoplastic material 33, for example COP or COC resin, is melted and delivered to the second injection mold, where it flows over top of the surface of the intermediate polymeric body and the electronic inlay 1 positioned thereon to form the molded syringe barrel. The final molded syringe barrel is then cooled and removed from the second injection mold. As described above, the result is a syringe barrel having the electronic inlay fully embedded and enclosed by a portion of the wall, e.g. wall 11, transition region 13, flange 14, or hub portion 12.

By incorporating the electronic inlay 1 into the syringe barrel in this manner, the dimensions of the syringe barrel, and in particular the portion of the syringe barrel in which the inlay is embedded and enclosed, may be repeatably produced within tight tolerances and with high uniformity. This is of particular importance when dealing with small volume syringe barrels, such as those in which the lumen has a nominal fill volume between 0.25 and 10 mL, optionally between 0.5 and 5 mL, optionally between 0.5 and 1 mL, optionally 0.5 mL, optionally 1 mL, optionally 2.25 mL. For instance, dimensional and surface uniformity are of great importance for maintaining container closure integrity (CCI).

Further, because the electronic inlay 1 is fully enclosed by the polymeric wall of the syringe barrel 11 and thus no portion of the electronic inlay is exposed on either the interior or exterior surfaces of the syringe barrel. Nor, in the case of a staked needle syringe, does the electronic inlay 1 contact the needle.

Incorporation of an electronic inlay 1 into the hub portion 12 of a staked needle syringe barrel is complicated by the specific structural arrangement of that element, including for instance the requirement that it secure the proximal portion of the needle therein, and the fact that the end of a needle holder typically forms part of the mold. Accordingly, the molding process must be specially adapted when incorporating an electronic inlay 1 in the hub portion 12 of a staked needle syringe barrel. In some embodiments, for example, the injection mold may comprise independently openable elements surrounding at least part of the hub portion 12 and which may be opened to allow for placement of the electronic inlay 1 without movement of the needle holder or the mold core. In some embodiments, the thickness of the first polymer layer or the intermediate polymeric body is carefully controlled such that, when cooled, the needle is secured therein, yet without taking up too much of the overall thickness of hub portion 12 such that the second polymeric layer is insufficient to completely cover and encapsulate the electronic inlay 1.

The electronic inlay may be relatively small, such that it does not require any significant changes in the dimensions of an existing vial, syringe, cartridge, etc. In some embodiments, for example, the electronic inlay may have a length of 5 mm or less, 4 mm or less, 3 mm or less, 2 mm or less, or 1 mm or less, and a width of 5 mm or less, 4 mm or less, 3 mm or less, 2 mm or less, or 1 mm or less. Embodiments of the electronic inlay may have a thickness of 500 microns or less, 400 microns or less, 300 microns or less, 200 microns or less, 100 microns or less, or 50 microns or less.

The electronic inlay may also be configured to withstand sterilization of the pharmaceutical package, such as sterilization by irradiation (e.g., e-beam or gamma) and gas (e.g., ethylene oxide (EtO), vaporized hydrogen peroxide (VHP), etc.).

The electronic inlay may also be configured to withstand PECVD coating of one or more surfaces of the vessel wall, such as is described for example in Trilayer Coated Pharmaceutical Packaging, U.S. Pat. No. 9,554,968, and PECVD Lubricity Vessel Coating, Coating Process And Apparatus Providing Different Power Levels in Two Phases, U.S. Pat. No. 9,863,042, the entireties of which are incorporated herein by reference. These coating processes may involve the electronic inlay being subjected to an electric field, for instance.

In some embodiments, a syringe barrel with an integrated staked needle and an embedded electronic component may be prepared. Another aspect of the technology disclosed here is a method of making a syringe with an electronic component and an integrated, staked needle by injection molding at least a portion of the barrel around the needle to secure and seal the needle in place. At least a portion of the needle is inserted into the mold prior to injecting the plastic or other material to form the syringe body. As the plastic cools in the mold cavity, the plastic syringe body bonds to the needle and forms a permanent attachment between the needle and the syringe. The bond between the needle and the syringe optionally is moisture tight, liquid tight, sufficient to maintain sterility, and can hold a vacuum.

In some embodiments, a vial with an embedded electronic component may be prepared. Bottles or vials may typically be formed using blow molding. Blow molding is a manufacturing process by which hollow plastic parts, e.g., bottles or vials (having a comparatively narrow neck and/or opening), are formed. In general, there are three types of blow molding: (1) extrusion blow molding; (2) injection blow molding; and (3) injection stretch blow molding. In any type of blow molding, the process begins with providing molten plastic and forming it into a parison or preform. The parison is a tube-like piece of plastic with an opening in one end through which compressed air can pass. The parison is clamped into a mold and air is blown into it. The air pressure pushes the plastic out (almost like blowing a balloon) to match the contours of the mold, thus forming a finished part once it has cooled. After the vessel has cooled and hardened, the mold is opened and the part ejected.

Extrusion blow molding is a process that is substantially as described before but also requires spin trimming, which is an additional step involving cutting excess material away. Extrusion blow molded parts are known to have low strength and are consequently not desirable for most containers. Also, the additional processing steps involved render extrusion blow molding unfavorable for making lyophilization vials.

In the standard injection blow molding (IBM) process, the polymer is injection molded onto a core pin; then the core pin is rotated to a blow molding station to be inflated and cooled. This is the least-used of the three blow molding processes, and is typically used to make small medical and single serve bottles. The process is divided into three steps: injection, blowing and ejection. The injection blow molding machine is based on an extruder barrel and screw assembly which melts the polymer. The molten polymer is fed into a hot runner manifold where it is injected through nozzles into a heated cavity and core pin. The cavity mold forms the external shape of the vessel and is clamped around a core rod which forms the internal shape of the preform. The preform consists of a fully formed bottle/jar neck with a thick tube of polymer attached, which will form the body, similar in appearance to a test tube with a threaded neck. An example of such a preform may be found in U.S. Pat. Pub. No. 2009/0220809. The preform mold opens and the core rod is rotated and clamped into the hollow, chilled blow mold. The end of the core rod opens and allows compressed air into the preform, which inflates it to the finished article shape. After a cooling period the blow mold opens and the core rod is rotated to the ejection position. Typically, injection blow molding only suits small capacity bottles as it is difficult to control the base center during blowing. Additionally, there is no increase in barrier strength as the material is not biaxially stretched. Accordingly, standard injection blow molding methods are undesirable for most containers and vessels due to limited uses or product configurations, barrier strength limitations, and other manufacturing disadvantages.

Traditional injection stretch blow molding (ISBM) is typically carried out using one of two different methods, namely single-stage and two-stage.

In the two-stage injection stretch blow molding process, the plastic is first molded into a preform using the injection molding process. These preforms are produced with the necks of the bottles, optionally including threads on one end. These preforms are packaged, and fed later (after cooling) into a reheat stretch blow molding machine. In the ISBM process, the preforms are heated above their glass transition temperature, then blown using high-pressure air into bottles using metal blow molds. The preform may also be stretched with a core rod or mandrel as part of the process.

FIG. 5 shows a schematic illustration of the steps involved in stretch blow molding according to an aspect of the disclosed concept. It is noted that the final vial shown in FIG. 5 is intended to be merely illustrative of the process and does not precisely and intricately depict all structural features of vial. As shown in step 41, the process may involve an initial step of: providing a plastic resin 33 (e.g., cyclin olefin polymer), melting the resin and delivering the melt to an injection mold, and molding a preform 30 from the resin. In a next step 42, the heated preform 30 is stretched optionally within the blow mold mold using a mandrel. When in the blow mold, the preform is stretched past the bottom (i.e., the base mold is not yet in position for blowing such that the mold is not in blow position). In a last step 43, gas is blown into the stretched heated preform while the mold parts and base mold are collectively in blow position (i.e., the base mold is pushed up from its previous position during the stretch step) to form the final shape of the vial. Moving the base mold up in this way after stretching and then blowing gas helps to optimize material distribution, especially in the corners. Blow pressure may be adjusted and controlled during steps 42 and 43. For example, optionally low pressure blow may be utilized during the stretch step 42 to help distribute the material of the preform out. Gas may be blown at high pressure when the base mold is in position, after the desired shape of the vial is partially formed. Optionally, a four component injection mold is used to help optimize the process and result in improved material distribution.

During the stretching step, the mandrel may be controlled pneumatically or by servo. Servo may be preferred because it provides more precise control in stretch speed and position of the mandrel. Also, a servo may optionally be used to monitor plastic temperature and adjust speed profile to help achieve a vial with desirable dimensions and tolerance necessary for thermal efficiency.

Applicant has found that the lower stretch ratio produced using injection stretch blow molding compared to other forms of blow molding enables better control of the part side wall thickness variation. Dimensional control and tolerances of the resulting part can improve the thermal efficiency of the container or vessel (e.g., vial). Minimizing side wall thickness variation facilitates more consistent heat transfer during a freeze drying (lyophilization) cycle. Notably, consistent side wall thickness measured radially (i.e., 360° around a central axis of the vial) appears to be more important than consistency of wall thickness measured axially (i.e., wall thickness at the top of the vial versus that near the bottom). In addition to advantages relating to thermal efficiency, sidewall thickness consistency of a vial produced according to the injection stretch blow molding method of the disclosed concept results in improved optical properties. Such properties in parenteral containers is necessary to permit visual inspection through the clear container for any foreign contamination. Side wall thickness inconsistency can create optical distortion, which limits one's ability to visually inspect the contents of the vial. The methods and vials of the disclosed concept reduce or eliminate this problem of the prior art.

It has further been found that density of the polymer vials made according to the above-noted process is much more consistent and precisely controlled than that of glass vials. Variations in density can affect the cycle time for lyophilization. Thus, the more consistent density of the vials according to the disclosed concept provide improved consistency in the lyophilization process.

By providing vessels having electronic components, e.g. RFID tags, incorporated therein during manufacture of the vessel, embodiments of the present disclosure may also enable a more efficient process for identifying rejected vessels, e.g. vials, syringe barrels, cartridges, or the like. Conventionally, when it is identified that one or more empty vessels or one or more filled and sealed vessels (i.e. pharmaceutical products) need to be rejected—e.g. due to one or more process parameters having been found to not be satisfied—it may be difficult to determine which individual vessels of a batch or a portion of a batch need to be rejected. This may lead to an entire batch or a significant portion of a batch being discarded. By providing each individual vessel with an electronic component, e.g. RFID tag, having a unique identifier, embodiments of the present disclosure enable the identification of individual vessels or pharmaceutical products for rejection, thereby limiting the amount of product that is discarded.

### Example Embodiments

### Pharmaceutical Packages

1. A pharmaceutical package comprising:
a vessel defining a lumen,
a pharmaceutical solution within the lumen,
a closure, and
an electronic component attached to the vessel and configured to provide the package with the ability to be traced; and/or detect and/or record compliance or non-compliance with one or more
storage conditions; and/or provide information regarding one or more administration parameters.

2. The pharmaceutical package of any preceding embodiment, wherein the vessel is a syringe barrel, a cartridge, or a vial.

3. The pharmaceutical package of any preceding embodiment, wherein the electronic component is an RFID tag, optionally an RFID tag that utilizes one or more frequencies within the range of 865 to 928 MHz, optionally an RFID tag that utilizes a frequency of about 13.56 MHz.

4. The pharmaceutical package of any preceding embodiment, wherein the electronic component comprises an integrated circuit.

5. The pharmaceutical package of any preceding embodiment, wherein the electronic component comprises data storage.

6. The pharmaceutical package of any preceding embodiment, wherein the vessel comprises at least one wall made of a thermoplastic material.

7. The pharmaceutical package of any preceding embodiment, wherein the thermoplastic material comprises one or more of the following: polypropylene, polyethylene, COP, COC, or CBC.

8. The pharmaceutical package of any preceding embodiment, wherein the electronic component is embedded in a thermoplastic wall of the vessel.

9. The pharmaceutical package of any preceding embodiment, wherein the electronic component is overmolded into a wall of the vessel.

10. The pharmaceutical package of any preceding embodiment, wherein the thermoplastic material that makes up at least a portion of the vessel completely or substantially completely surrounds the electronic component.

11. The pharmaceutical package of any preceding embodiment, wherein no part of the electronic component is on the exterior surface of the vessel.

12. The pharmaceutical package of any preceding embodiment, wherein the package is a pre-filled syringe and wherein the electronic component is embedded in a hub portion of the syringe barrel.

13. The pharmaceutical package of any preceding embodiment, wherein the package is a pre-filled syringe and wherein the electronic component is embedded in a transition region between a main body of the syringe barrel and a hub portion of the syringe barrel.

14. The pharmaceutical package of any preceding embodiment, wherein the hub portion is a needle hub or a Luer hub.

15. The pharmaceutical package of any preceding embodiment, wherein the closure comprises a rigid needle shield or a Luer cap, and wherein the electronic component is visually concealed by the closure.

16. The pharmaceutical package of any preceding embodiment, wherein the package is a pre-filled syringe and wherein the electronic component is embedded in a main body of the syringe barrel adjacent a rear flange.

17. The pharmaceutical package of any preceding embodiment, wherein the package is a filled vial, and wherein the electronic component is embedded in a neck portion of the vial, optionally in a thickened flange region.

18. The pharmaceutical package of any preceding embodiment, wherein the closure comprises a stopper and a crimp, and wherein the electronic component is visually concealed by the closure.

19. The pharmaceutical package of any preceding embodiment, wherein the package is a filled cartridge, and wherein the electronic component is embedded in a needle attachment portion of the cartridge.

20. The pharmaceutical package of any preceding embodiment, wherein the closure comprises a cap and the electronic component is visually concealed by the closure.

21. The pharmaceutical package of any preceding embodiment, further comprising a label and wherein the electronic component is embedded in a portion of the vessel that is visually concealed by the label.

22. The pharmaceutical package of any preceding embodiment, wherein the package is a filled cartridge and the electronic component is configured to be directly read by a delivery device, optionally an auto-injector or an injection pen.

23. The pharmaceutical package of any preceding embodiment, wherein the electronic component is visually concealed by a portion of the package.

24. The pharmaceutical package of any preceding embodiment, wherein the package is configured so that the vessel may also be traced prior to filling of the lumen with the pharmaceutical solution and sealing of the lumen with the closure.

25. The pharmaceutical package of any preceding embodiment, wherein the electronic component is detectable by automated visual inspection of the vessel in an empty state.

26. The pharmaceutical package of any preceding embodiment, wherein the electronic component is configured to be written to using an external writer.

27. The pharmaceutical package of any preceding embodiment, wherein the pharmaceutical solution is an injectable drug-containing solution.

28. The pharmaceutical package of any preceding embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

29. The pharmaceutical package of any preceding embodiment, wherein the electronic component has a thickness of 500 microns or less, optionally 400 microns or less, optionally 300 microns or less, optionally 200 microns or less, optionally 100 microns or less, optionally 50 microns or less.

30. The pharmaceutical package of any preceding embodiment, wherein the pharmaceutical package has been subjected to sterilization, optionally sterilization by irradiation, optionally sterilization by gas.

31. The pharmaceutical package of any preceding embodiment, wherein the electronic component is configured to withstand sterilization of the pharmaceutical package, optionally sterilization by irradiation, optionally sterilization by gas.

32. The pharmaceutical package of any preceding embodiment, in which the electronic component comprises a temperature sensor, optionally in which the electronic component is a passive RFID temperature sensor.

33. The pharmaceutical package of any preceding embodiment, in which the pharmaceutical solution within the lumen must be maintained within a defined temperature range and the electronic component is configured to detect and register a deviation from that temperature range.

34. The pharmaceutical package of any preceding embodiment, in which the electronic component comprises a pressure sensor, optionally in which the electronic component is a passive RFID pressure sensor.

35. The pharmaceutical package of any preceding embodiment, in which the pharmaceutical solution within the lumen must be maintained within a defined pressure range and the electronic component is configured to detect and register a deviation from that pressure range.

36. The pharmaceutical package of any preceding embodiment, in which the pharmaceutical solution is to be administered at a defined dispense rate or within a defined range of acceptable dispense rates and the electronic component contains information regarding the defined dispense rate or defined range of acceptable dispense rates.

37. The pharmaceutical package of any preceding embodiment, in which the information regarding the defined dispense rate or defined range of acceptable dispense rates is configured to be read by a delivery device, optionally an auto-injector or injection pen.

38. The pharmaceutical package of any preceding embodiment, in which the pharmaceutical solution is to be administered using a defined needle insertion depth or defined range of acceptable needle insertion depths and the electronic component contains information regarding the defined needle insertion depth or defined range of acceptable needle insertion depths.

39. The pharmaceutical package of any preceding embodiment, in which the information regarding the defined needle insertion depth or defined range of acceptable needle insertion depths is configured to be read by a delivery device, optionally an auto-injector or injection pen.

40. The pharmaceutical package of any preceding embodiment, in which the electronic component is configured to register an interrogation event into a database.

41. An auto-injector or injection pen comprising the cartridge or syringe of any preceding embodiment, wherein the auto-injector or injection pen comprises a reader configured to read the electronic component of the cartridge or syringe.

42. The auto-injector or injection pen of embodiment 41, wherein the auto-injector or injection pen is configured to adjust one or more injection settings in response to information obtained from the electronic component.

43. The pharmaceutical package of any preceding embodiment, in which the inner surface, the outer surface, or both of the wall in which the electronic component is embedded are identical or substantially identical to those of the wall of the same vessel prepared without the electronic component.

44. The pharmaceutical package of any preceding embodiment, in which the inner surface, the outer surface, or both of the wall in which the electronic component is embedded are free of surface irregularities, such as those caused by insertion of an electronic component.

45. The pharmaceutical package of any preceding embodiment, in which the electronic component comprises information, optionally identification information, stored directly on the component.

46. The pharmaceutical package of any preceding embodiment, in which the electronic component is readable without connection to a computing network such as a cloud-computing network.

47. The pharmaceutical package of any preceding embodiment, in which the electronic component is writable without connection to a computing network such as a cloud-computing network.

### Vessels (e.g., Prior to Filling)

A1. A vessel having an electronic component attached to the vessel and configured to provide both the unfilled vessel and the pharmaceutical package that results from filling a lumen of the vessel with a pharmaceutical solution and then sealing the lumen with the ability to be traced.

A2. The vessel of embodiment A1, wherein the vessel is a syringe barrel, a vial, or a cartridge.

A3. A syringe barrel having an electronic component attached to the syringe barrel and configured to provide both the unfilled syringe barrel and the pharmaceutical package that results from filling a lumen of the syringe barrel with a pharmaceutical solution and then sealing the lumen with the ability to be traced.

A4. A vial having an electronic component attached to the vial and configured to provide both the unfilled vial and the pharmaceutical package that results from filling a lumen of the vial with a pharmaceutical solution and then sealing the lumen with the ability to be traced.

A5. A cartridge having an electronic component attached to the cartridge and configured to provide both the unfilled cartridge and the pharmaceutical package that results from filling a lumen of the cartridge with a pharmaceutical solution and then sealing the lumen with the ability to be traced.

A6. The vessel, syringe barrel, vial or cartridge of any preceding embodiment, wherein the electronic component is an RFID tag, optionally an RFID tag that utilizes one or more frequencies within the range of 865 to 928 MHz, optionally an RFID tag that utilizes a frequency of about 13.56 MHz.

A7. The vessel, syringe barrel, vial or cartridge of any preceding embodiment, wherein the electronic component comprises an integrated circuit.

A8. The vessel, syringe barrel, vial or cartridge of any preceding embodiment, wherein the electronic component comprises data storage.

A9 The vessel, syringe barrel, vial or cartridge of any preceding embodiment, wherein the vessel, syringe barrel, vial or cartridge comprises at least one wall made of a thermoplastic material.

A10. The vessel, syringe barrel, vial or cartridge of any preceding embodiment, wherein the thermoplastic material comprises one or more of the following: polypropylene, polyethylene, COP, COC, or CBC.

A11. The vessel, syringe barrel, vial or cartridge of any preceding embodiment, wherein the electronic component is embedded in a thermoplastic wall of the vessel, syringe barrel, vial or cartridge.

A12. The vessel, syringe barrel, vial or cartridge of any preceding embodiment, wherein the electronic component is overmolded into a wall of the vessel, syringe barrel, vial or cartridge.

A13. The vessel, syringe barrel, vial or cartridge of any preceding embodiment, wherein the thermoplastic material that makes up at least a portion of the vessel, syringe barrel, vial or cartridge completely or substantially completely surrounds the electronic component.

A14. The vessel, syringe barrel, vial or cartridge of any preceding embodiment, wherein no part of the electronic component is on the exterior surface of the vessel, syringe barrel, vial or cartridge.

A15. The syringe barrel of any preceding embodiment, wherein the electronic component is embedded in a hub portion of the syringe barrel.

A16. The syringe barrel of any preceding embodiment, wherein the electronic component is embedded in a transition region between a main body of the syringe barrel and a hub portion of the syringe barrel.

A17. The syringe barrel of any preceding embodiment, wherein the hub portion is a needle hub or a Luer hub.

A18. The syringe barrel of any preceding embodiment, wherein the syringe barrel is configured so that the electronic component is visually concealed by a rigid needle shield or Luer cap.

A19. The syringe barrel of any preceding embodiment, wherein the electronic component is embedded in a main body of the syringe barrel adjacent a rear flange.

A20. The vial of any preceding embodiment, wherein the electronic component is embedded in a neck portion of the vial, optionally in a thickened flange region.

A21. The vial of any preceding embodiment, wherein the vial is configured so that the electronic component is visually concealed by a stopper and crimp closure.

A22. The cartridge of any preceding embodiment, wherein the electronic component is embedded in a needle attachment portion of the cartridge.

A23. The cartridge of any preceding embodiment, wherein the cartridge is configured so that the electronic component is visually concealed by a cap placed on the needle attachment portion.

A24. The vessel, syringe barrel, vial or cartridge of any preceding embodiment, further comprising a label and wherein the electronic component is embedded in a portion of the vessel, syringe barrel, vial or cartridge that is visually concealed by the label.

A25. The cartridge of any preceding embodiment, wherein cartridge is configured so that the electronic component is directly readable by a delivery device, optionally an auto-injector or an injection pen.

A26. The vessel, syringe barrel, vial or cartridge of any preceding embodiment, wherein the vessel, syringe barrel, vial or cartridge is configured so that the electronic component is visually concealed by a portion of the finished pharmaceutical package.

A27. The vessel, syringe barrel, vial or cartridge of any preceding embodiment, wherein the electronic component is detectable by automated visual inspection of the vessel, syringe barrel, vial or cartridge in an empty state.

A28. The vessel, syringe barrel, vial or cartridge of any preceding embodiment, wherein the electronic component is configured to be written to using an external writer.

A29. The vessel, syringe barrel, vial or cartridge of any preceding embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

A30. The vessel, syringe barrel, vial or cartridge of any preceding embodiment, wherein the electronic component has a thickness of 500 microns or less, optionally 400 microns or less, optionally 300 microns or less, optionally 200 microns or less, optionally 100 microns or less, optionally 50 microns or less.

A31. The vessel, syringe barrel, vial or cartridge of any preceding embodiment, wherein the vessel, syringe barrel, vial or cartridge is configured such that the electronic component can withstand sterilization, optionally sterilization by e-beam or gamma irradiation, optionally sterilization by Eto or VHP gas.

A32. The vessel, syringe barrel, vial or cartridge of any preceding embodiment, in which the electronic component comprises a temperature sensor, optionally in which the electronic component is a passive RFID temperature sensor.

A33. The vessel, syringe barrel, vial or cartridge of any preceding embodiment, in which the electronic component comprises a pressure sensor, optionally in which the electronic component is a passive RFID pressure sensor.

A34. The vessel, syringe barrel, vial or cartridge of any preceding embodiment, in which the electronic component contains information regarding a defined dispense rate or defined range of acceptable dispense rates.

A35. The vessel, syringe barrel, vial or cartridge of any preceding embodiment, in which the information regarding the defined dispense rate or defined range of acceptable dispense rates is configured to be read by a delivery device, optionally an auto-injector or injection pen.

A36. The vessel, syringe barrel, vial or cartridge of any preceding embodiment, in which the electronic component contains information regarding a defined needle insertion depth or defined range of acceptable needle insertion depths.

A37. The vessel, syringe barrel, vial or cartridge of any preceding embodiment, in which the information regarding the defined needle insertion depth or defined range of acceptable needle insertion depths is configured to be read by a delivery device, optionally an auto-injector or injection pen.

A38. The vessel, syringe barrel, vial or cartridge of any preceding embodiment, in which the electronic component is configured to register an interrogation event into a database.

A39. The vessel, syringe barrel, vial or cartridge of any preceding embodiment, in which the inner surface, the outer surface, or both of the wall in which the electronic component is embedded and enclosed are identical or substantially identical to those of the wall of the same vessel, syringe barrel, vial or cartridge prepared without the electronic component.

A40. The vessel, syringe barrel, vial or cartridge of any preceding embodiment, in which the inner surface, the outer surface, or both of the wall in which the electronic component is embedded and enclosed are free of surface irregularities, such as those caused by insertion of an electronic component.

A41. The vessel, syringe barrel, vial or cartridge of any preceding embodiment, in which the electronic component comprises information, optionally identification information, stored directly on the component.

A42. The vessel, syringe barrel, vial or cartridge of any preceding embodiment, in which the electronic component is readable without connection to a computing network such as a cloud-computing network.

A42. The vessel, syringe barrel, vial or cartridge of any preceding embodiment, in which the electronic component is writable without connection to a computing network such as a cloud-computing network.

### Molding/Inspection Processes

B1. A method of preparing a vessel having an electronic component embedded in a thermoplastic material that makes up at least a portion of the vessel, the method comprising:
inserting the electronic component in a mold
injecting thermoplastic material into the mold to form the vessel or at least a portion of the vessel,
   wherein the thermoplastic material surrounds or substantially surrounds the electronic component, thereby embedding the electronic component within a portion of the vessel.

B2. A method of preparing a vessel having an electronic component embedded in a thermoplastic material that makes up at least a portion of the vessel, the method comprising:
providing a preform having an electronic component embedded therein, optionally in which the electronic component is embedded in an upper portion of the preform that corresponds with a neck of the vessel,
injection blow molding or injection stretch blow molding the preform to produce the vessel.

B3. The method of any preceding embodiment, wherein the vessel is a syringe barrel, medical cartridge, or vial.

B4. A method of preparing a syringe barrel having an electronic component embedded in a thermoplastic material that makes up at least a portion of the syringe barrel, the method comprising:
inserting the electronic component in a mold
injecting thermoplastic material into the mold to form the syringe barrel or at least a portion of the syringe barrel,
   wherein the thermoplastic material surrounds or substantially surrounds the electronic component, thereby embedding the electronic component within a portion of the syringe barrel.

B4A. A method of preparing a syringe barrel having an electronic component embedded in a thermoplastic material that makes up at least a portion of the syringe barrel, the method comprising:
injection molding a first polymeric layer;
cooling the first polymeric layer, e.g. to a temperature below its glass transition temperature;
positioning the electronic component on a surface of the first polymeric layer; and
injection molding a second polymeric layer, such that the second polymeric layer covers the electronic component.

B5. A method of preparing a vial having an electronic component embedded in a thermoplastic material that makes up at least a portion of the vial, the method comprising:
providing a preform having an electronic component embedded therein, optionally in which the electronic component is embedded in an upper portion of the preform that corresponds with the neck of the vial,
injection blow molding or injection stretch blow molding the preform to produce the vial.

B6. The method of any previous embodiment, further comprising inspecting the vessel, syringe barrel, or vial to ensure that presence, positioning, and/or functioning of the electronic component.

B7. The method of any previous embodiment, wherein the inspecting comprises an automated machine-operated visual inspection of the vessel, syringe barrel, or vial to identify the presence of the electronic component.

B8. The method of any previous embodiment, wherein the machine-operated visual inspection also determines whether the electronic component is in an acceptable location within the vessel, syringe barrel, or vial.

B9. The method of any previous embodiment, wherein the inspecting comprises using a remote reader to ensure that the electronic component is readable.

B10. The method of any previous embodiment, wherein the electronic component is an RFID tag, optionally an RFID tag that utilizes one or more frequencies within the range of 865 to 928 MHz, optionally an RFID tag that utilizes a frequency of about 13.56 MHz.

B11. The method any preceding embodiment, wherein the electronic component comprises an integrated circuit.

B12. The method of any preceding embodiment, wherein the electronic component comprises data storage.

B13. The method of any preceding embodiment, wherein the thermoplastic material comprises one or more of the following: polypropylene, polyethylene, COP, COC, or CBC.

B14. The method of any preceding embodiment, wherein no part of the electronic component is on the exterior surface of the vessel, syringe barrel, vial or cartridge, and optionally wherein no part of the electronic component is on an interior surface of the vessel, syringe barrel, vial, or cartridge.

B15. The method of any preceding embodiment, wherein the electronic component is embedded in a hub portion of the syringe barrel.

B16. The method of any preceding embodiment, wherein the electronic component is embedded in a transition region between a main body of the syringe barrel and a hub portion of the syringe barrel.

B17. The method of any preceding embodiment, wherein the hub portion is a needle hub or a Luer hub.

B18. The method of any preceding embodiment, wherein the syringe barrel is configured so that the electronic component is visually concealed by a rigid needle shield or Luer cap.

B19. The method of any preceding embodiment, wherein the electronic component is embedded in a main body of the syringe barrel adjacent a rear flange.

B20. The method of any preceding embodiment, wherein the electronic component is embedded in a neck portion of the vial, optionally a thickened flange region.

B21. The method of any preceding embodiment, wherein the vial is configured so that the electronic component is visually concealed by a stopper and crimp closure.

B22. The method of any preceding embodiment, wherein the vessel is a cartridge and the electronic component is embedded in a needle attachment portion of the cartridge.

B23. The method of any preceding embodiment, wherein the cartridge is configured so that the electronic component is visually concealed by a cap placed on the needle attachment portion.

B24. The method of any preceding embodiment, wherein the vessel, syringe barrel, vial or cartridge further comprises a label and wherein the electronic component is embedded in a portion of the vessel, syringe barrel, vial or cartridge that is visually concealed by the label.

B25. The method of any preceding embodiment, wherein the cartridge is configured so that the electronic component is directly readable by a delivery device, optionally an auto-injector or an injection pen.

B26. The method of any preceding embodiment, wherein the vessel, syringe barrel, vial or cartridge is configured so that the electronic component is visually concealed by a portion of a finished pharmaceutical package.

B27. The method of any preceding embodiment, wherein the electronic component is detectable by automated visual inspection of the vessel, syringe barrel, vial or cartridge in an empty state.

B28. The method of any preceding embodiment, wherein the electronic component is configured to be written to using an external writer.

B29. The method of any preceding embodiment, wherein the electronic component has a length of 5 mm or less, optionally 4 mm or less, optionally 3 mm or less, optionally 2 mm or less, optionally 1 mm or less, and a width of 5 mm or less, optionally 4 mm or less, optionally 3 mm or less, optionally 2 mm or less, optionally 1 mm or less.

B30. The method of any preceding embodiment, wherein the electronic component has a thickness of 500 microns or less, optionally 400 microns or less, optionally 300 microns or less, optionally 200 microns or less, optionally 100 microns or less, optionally 50 microns or less.

B31. The method of any preceding embodiment, wherein the vessel, syringe barrel, vial or cartridge is configured such that the electronic component can withstand sterilization, optionally sterilization by e-beam or gamma irradiation, optionally sterilization by Eto or VHP gas.

B32. The method of any preceding embodiment, in which the inner surface, the outer surface, or both of the wall of the vessel, syringe barrel, vial or cartridge in which the electronic component is embedded and enclosed are identical or substantially identical to those of the wall of the same vessel, syringe barrel, vial or cartridge prepared without the electronic component.

B33. The method of any preceding embodiment, in which the inner surface, the outer surface, or both of the wall of the vessel, syringe barrel, vial or cartridge in which the electronic component is embedded and enclosed are free of surface irregularities, such as those caused by insertion of an electronic component.

B34. The method of any preceding embodiment, in which the step of providing a preform having an electronic component embedded therein comprises
injection molding a first polymeric layer;
cooling the first polymeric layer, e.g. to a temperature below its glass transition temperature;
positioning the electronic component on a surface of the first polymeric layer; and
injection molding a second polymeric layer, such that the second polymeric
layer covers the electronic component.

B35. The method of embodiment B34, in which the step of providing a preform having an electronic component embedded therein further comprises:
transferring a polymeric body resulting from cooling of the first polymer layer from a first injection mold to a second injection mold; and
injection molding the second polymeric layer in the second injection mold.

B36. The method of any previous embodiment, in which the electronic component is positioned at a portion of the preform so that the step of injection blow molding or injection stretch blow molding to produce the vessel or vial places substantially no stresses on the electronic component.

B37. The method of embodiment B4A, in which the method of preparing a syringe barrel having an electronic component embedded in a thermoplastic material that makes up at least a portion of the syringe barrel further comprises:
transferring a polymeric body resulting from cooling of the first polymer layer from a first injection mold to a second injection mold; and
injection molding the second polymeric layer in the second injection mold.

B38. The method of any preceding embodiment, wherein the syringe barrel resulting from the molding process comprises a needle, the proximal end of which is embedded in the needle hub.

B39. The method of any preceding embodiment, wherein the injection molding of the first polymer layer, the positioning of the electronic component, and the injection molding of the second polymer layer are performed without moving the needle or mold core relative to one another.

B40. The method of any preceding embodiment, wherein a portion of an injection mold surrounding the needle hub opens to enable the positioning of the electronic component.

B41. The method of any preceding embodiment, wherein the needle is securely embedded a polymeric body resulting from cooling of the first polymer layer.

B42. The method of any preceding embodiment, wherein the needle is inserted into the second injection mold, optionally in which the needle is not present in the first injection mold.

B43. The method any preceding embodiment, in which the electronic component comprises information, optionally identification information, stored directly on the component.

B44. The method of any preceding embodiment, in which the electronic component is readable without connection to a computing network such as a cloud-computing network.

B45. The method of any preceding embodiment, in which the electronic component is writable without connection to a computing network such as a cloud-computing network.

### Methods of Use

C1. A method of monitoring whether or not a pharmaceutical package is maintained within defined storage conditions, comprising:
providing a pharmaceutical package according to any of the preceding embodiments;
registering any non-compliance events detected by the temperature sensor (if applicable), the pressure sensor (if applicable), or both.

C1a. A method of facilitating administration of the pharmaceutical solution contained within the lumen of a pharmaceutical package according to one of the preceding embodiments comprising:
interrogating the electronic component prior to administration, thereby obtaining information regarding whether there has been a non-compliance event detected by the temperature sensor (if applicable), the pressure sensor (if applicable), or both, and
optionally rejecting the pharmaceutical package if a non-compliance event was detected.

C1b. The method of embodiment C1a, wherein the interrogating is performed by a drug delivery device, optionally an auto-injector or injection pen.

C1c. The method of embodiment C1b, wherein the drug delivery device is configured to reject the pharmaceutical package if a non-compliance event is detected.

C2. A method of facilitating proper administration of the pharmaceutical solution contained within the lumen of a pharmaceutical package according to one of the preceding embodiments comprising:
interrogating the electronic component prior to administration, thereby obtaining the information regarding one or more administration parameters; and
administering the pharmaceutical solution in accordance with the one or more administration parameters.

C3. The method of embodiment C2, wherein the interrogating and administering is performed by a drug delivery device, optionally an auto-injector or injection pen.

C4. The method of embodiment C3, further comprising adjusting one or more settings of the drug delivery device to comply with the one or more administration parameters, optionally wherein the adjusting is performed automatically by the drug delivery device.

C5. A method of monitoring administration of pharmaceutical packages, e.g. as part of a clinical trial, comprising:
providing pharmaceutical packages according to any of the preceding embodiments;
registering, to a database:
   the date and time of interrogations of the electronic components, and/or
   non-compliance events detected by the temperature sensor (if applicable), the pressure sensor (if applicable), or both.

C6. A method of providing a pharmaceutical product with identification information, comprising:
obtaining a vessel, syringe barrel, vial or cartridge according to any of the preceding embodiments, optionally wherein the electronic component comprises identification information relating to the vessel, syringe barrel, vial or cartridge;
filling the vessel with a pharmaceutical formulation; and
writing information relating to the pharmaceutical formulation to the electronic component, optionally without connection to a computing network such as a cloud-computing network.

C7. The method of embodiment C6, wherein the identification information relating to the vessel, syringe barrel, vial or cartridge is readable directly from the electronic component.

1. A vessel having an electronic component attached to the vessel;
wherein the vessel is a syringe barrel, vial, or cartridge having a thermoplastic wall;
wherein the electronic component is embedded in the thermoplastic wall of the vessel; and
wherein the electronic component is configured to provide both the vessel in an unfilled state and a pharmaceutical package that results from filling a lumen of the vessel with a pharmaceutical solution and then sealing the lumen with the ability to be traced.

2.-5. (canceled)

6. The vessel of claim 1, wherein the electronic component is an RFID tag.

7. The vessel of claim 1, wherein the electronic component comprises an integrated circuit.

8. The vessel of claim 1, wherein the electronic component comprises data storage.

9. (canceled)

10. The vessel of claim 1, wherein the thermoplastic wall of the vessel comprises one or more of the following materials: polypropylene, polyethylene, COP, COC, or CBC.

11. (canceled)

12. The vessel of claim 1, wherein the electronic component is overmolded into the thermoplastic wall of the vessel.

13. The vessel of claim 1, wherein a thermoplastic material that makes up at least a portion of the vessel wall completely or substantially completely surrounds the electronic component.

14. The vessel of claim 1, wherein no part of the electronic component is present on an outer surface of the vessel wall.

15. The vessel of claim 1, further comprising a label, and wherein the electronic component is embedded in a portion of the vessel wall that is visually concealed by the label.

16. The vessel of claim 1, wherein the vessel is configured so that the electronic component is visually concealed by a portion of the pharmaceutical package.

17. The vessel of claim 1, wherein the electronic component is detectable by automated visual inspection of the vessel in an empty state.

18. The vessel of claim 1, wherein the electronic component is configured to be written to using an external writer.

19. The vessel of claim 1, wherein the vessel is configured such that the electronic component can withstand sterilization.

20. The vessel of claim 1, in which the electronic component comprises a temperature sensor.

21. The vessel of claim 1, in which the electronic component comprises a pressure sensor.

22. The vessel of claim 1, in which the electronic component is configured to register an interrogation event into a database.

23. The vessel of claim 1, in which an inner surface, an outer surface, or both, of the wall in which the electronic component is embedded are identical or substantially identical to those of the wall of the same vessel prepared without the electronic component.

24. The vessel of claim 1, in which the electronic component comprises identification information, stored directly on the component.

25. The vessel of claim 1, in which the electronic component is readable without connection to a computing network such as a cloud-computing network.

26. The vessel of claim 1, in which the electronic component is writable without connection to a computing network such as a cloud-computing network.
